SEQUENCE LISTING (30 sequences)

SEQ ID NO: 1 - DNA, 22 bp, artificial
  primer
cgcgtgaaggatgaaggttttc

SEQ ID NO: 2 - DNA, 20 bp, artificial
  primer
tcagttaaagcccagcaggc

SEQ ID NO: 3 - DNA, 21 bp, artificial
  primer
cgcggtaaaacgtaggtcaca

SEQ ID NO: 4 - DNA, 22 bp, artificial
  primer
ctgggacgttgtttctgagttt

SEQ ID NO: 5 - DNA, 22 bp, artificial
  primer
ggaatcttccacaatggacgca

SEQ ID NO: 6 - DNA, 22 bp, artificial
  primer
catggagttccactgtcctctt

SEQ ID NO: 7 - DNA, 22 bp, artificial
  primer
gtccatcgcttaacggtggatc

SEQ ID NO: 8 - DNA, 17 bp, artificial
  primer
accacctgtgaacccgc

SEQ ID NO: 9 - DNA, 18 bp, artificial
  primer
gcactccacctggggagt

SEQ ID NO: 10 - DNA, 22 bp, artificial
  primer
caaccttcctccgggttatcca

SEQ ID NO: 11 - DNA, 20 bp, artificial
  primer
ggcttgctggacaaatactg

SEQ ID NO: 12 - DNA, 20 bp, artificial
  primer
ctaggctcgtcagaaggatg

SEQ ID NO: 13 - DNA, 18 bp, artificial
  primer
ggtaggggagggtggaac

SEQ ID NO: 14 - DNA, 17 bp, artificial
  primer
gcggtcccgcgtgggtt

SEQ ID NO: 15 - DNA, 22 bp, artificial
  primer
ggagtgctaataccggatgtga

SEQ ID NO: 16 - DNA, 20 bp, artificial
  primer
ccgtggcttcctcgtttact

SEQ ID NO: 17 - DNA, 24 bp, artificial
  primer
cgtgaaggaagaagtatctcggta

SEQ ID NO: 18 - DNA, 21 bp, artificial
  primer
tcagttaccgtccagcaggcc

SEQ ID NO: 19 - DNA, 20 bp, artificial
  primer
  misc_feature (4)..(4): s is g or c
  misc_feature (9)..(9): y is t or c
  misc_feature (12)..(12): y is t or c
gtgstgcayggytgtcgtca

SEQ ID NO: 20 - DNA, 20 bp, artificial
  primer
  misc_feature (6)..(6): r is g or a
  misc_feature (12)..(12): r is g or a
acgtcrtccccrccttcctc

SEQ ID NO: 21 - DNA, 17 bp, artificial
  primer
attaccgcggctgctgg

SEQ ID NO: 22 - DNA, 15 bp, artificial
  primer
  misc_feature (14)..(14): r is g or a
acgggcggtgtgtrc

SEQ ID NO: 23 - DNA, 21 bp, artificial
  primer
  misc_feature (9)..(9): n is a, c, g, or t
cgggtgctncccactttcatg

SEQ ID NO: 24 - DNA, 18 bp, artificial
  primer
  misc_feature (2)..(2): n is a, c, g, or t
gntaccttgttacgactt

SEQ ID NO: 25 - DNA, 22 bp, artificial
  primer
  misc_feature (6)..(6): y is t or c
tacggytaccttgttacgactt

SEQ ID NO: 26 - DNA, 17 bp, artificial
  primer
  misc_feature (10)..(10): w is a or t
  misc_feature (15)..(15): r is g or a
aaggaggtgwtccarcc

SEQ ID NO: 27 - DNA, 18 bp, artificial
  primer
  misc_feature (12)..(12): m is a or c
agagtttgatcmtggctc

SEQ ID NO: 28 - DNA, 21 bp, artificial
  primer
gattctggctcaggatgaacg

SEQ ID NO: 29 - DNA, 20 bp, artificial
  primer
  misc_feature (12)..(12): m is a or c
agagtttgatcmtggctcag

SEQ ID NO: 30 - DNA, 20 bp, artificial
  primer
ccagcagccgcggtaatacg

(19) United States
(12) Patent Application Publication
(10) Pub. No.: US 2025/0082695 A1
(43) Pub. Date: Mar. 13, 2025

(54) USE OF GLYCEROL FOR INCREASING BUTYRATE PRODUCTION BY BACTERIA IN A CONSORTIUM

(71) Applicant: PHARMABIOME AG, SCHLIEREN (CH)

(72) Inventors: FABIENNE KURT, ZÜRICH (CH); CHRISTOPHE LACROIX, KILCHBERG (CH); TOMAS DE WOUTERS, ZÜRICH (CH)

(21) Appl. No.: 17/768,871

(22) PCT Filed: Oct. 14, 2020
(86) PCT No.: PCT/EP2020/078938

§ 371 Date: Apr. 14, 2022

(30) Foreign Application Priority Data
Oct. 15, 2019 (EP) 19203399.1

(51) Int. Cl. A61K 35/742; A61K 35/745; A61K 35/747; A61K 47/10; A61K 47/12; A61P 1/00

(52) U.S. Cl. CPC A61K 35/742; A61K 35/745; A61K 35/747; A61K 47/10; A61K 47/12; A61P 1/00

(57) ABSTRACT

The present invention provides utilizations and methods to increase production of butyrate by a consortium of living bacteria. The invention also relates to pharmaceutical compositions that comprise (i) at least one bacterial strain producing lactate, (ii) at least one bacterial strain consuming lactate, (iii) at least one bacterial strain producing butyrate and (iv) butyrate.

### CROSS-REFERENCE TO RELATED APPLICATION

This application is the U.S. national stage application of International Patent Application No. PCT/EP2020/078938, filed Oct. 14, 2020.

### REFERENCE TO SEQUENCE LISTING

The Sequence Listing for this application is labeled “Seq-List.txt” which was created on Apr. 5, 2022 and is 7 KB. The entire content of the sequence listing is incorporated herein by reference in its entirety.

### FIELD

The present invention relates to the field of microbiology. It provides uses and methods to increase production of butyrate by bacteria but also compositions comprising butyrate producing bacteria and butyrate.

### BACKGROUND OF THE INVENTION

Described as “an additional organ” and composed of more than thousand different bacterial species, the gut microbiota is known to play a beneficial role for the host by exerting many biological functions, such as nutrient absorption, maintenance of intestinal epithelium integrity, protection from pathogens or homeostasis of immune responses. Studies have shown that a persistent or transient imbalance of gut's microbial community, commonly referred to as dysbiosis, relates to several diseases, such as inflammatory bowel disease (IBD) or irritable bowel syndrome (IBS).

In addition, dysbiosis impairs the production of short-chain fatty acids (SCFA), organic fatty acids with one to six carbons that are produced within the intestinal lumen by bacterial fermentation of undigested dietary carbohydrates. Acetate, propionate, and butyrate are the most abundant SCFA produced in the gastrointestinal tract (GIT).

Among SCFA studies, there have been many investigations into the effects of butyrate and its potential applications in human health. Butyrate is known to have beneficial effects on epithelial barrier function and overall gut health. It is a cellular mediator regulating multiple functions of gut human and microbial cells including gene expression, immune modulation and oxidative stress reduction. Mammalian cells do not produce significant amounts of butyrate, so that the only significant sources are the microbiota and ingestion of dairy products.

One of the major problems in the application of butyrate is the difficulty in handling. The inventors address these drawbacks and herein provide a method to improve butyrate production by bacteria as well as compositions comprising butyrate producing bacteria.

### SUMMARY OF THE INVENTION

The inventors have discovered that the use of glycerol during the culture of butyrate producing bacteria promotes butyrate production by said bacteria.

In a first aspect, the invention concerns a pharmaceutical or nutraceutical composition comprising a consortium of bacteria comprising:
a. at least one bacterial strain producing lactate;
b. at least one bacterial strain consuming lactate; and
c. at least one bacterial strain producing butyrate, the production of butyrate of said bacterium being increased by at least 10% in the presence of glycerol; and
d. at least 10 mM butyrate, preferably at least 20 mM butyrate.

Preferably, such pharmaceutical or nutraceutical composition further comprises at least 5% of glycerol.

In particular, in such a composition, the butyrate producing bacterium is also a lactate consuming bacterium. Particularly, the bacterial strain producing butyrate is selected from the genera Eubacterium, Roseburia, Coprococcus, Faecalibacterium, Anaerostipes and Clostridium. Optionally, the bacterial strain producing butyrate is selected from the genus Eubacterium, preferably Eubacterium limosum, Eubacterium rectale, Eubacterium hallii or Eubacterium ramulus. Optionally, the bacterial strain producing butyrate is selected from the genus Roseburia, preferably Roseburia spp., Roseburia intestinalis, Roseburia hominis, Roseburia inulinivorans, or Roseburia faecis. Optionally, the bacterial strain producing butyrate is selected from the genus Coprococcus, preferably Coprococcus catus Coprococcus eutactus, or Coprococcus comes. Optionally, the bacterial strain producing butyrate is selected from the genus Faecalibacterium, preferably Faecalibacterium prausnitzii. Optionally, the bacterial strain producing butyrate is selected from the genus Anaerostipes, preferably Anaerostipes caccae or Anaerostipes hadrus. Optionally, the bacterial strain producing butyrate is selected from the genus Clostridium, preferably Clostridium indolis.

The pharmaceutical or nutraceutical composition according to the invention further comprises: (i) at least one lactate producing bacterium selected from the genera Lactobacillus, Streptococcus, Escherichia, Lactococcus, Enterococcus, Bifidobacterium, Collinsella, and Roseburia; and
at least one lactate consuming bacterium selected from the genera Anaerostipes, Eubacterium, Clostridium, Propionibacterium, Veillonella, Coprococcus and Megasphaera; and
(ii) optionally:
at least one bacterial strain selected from the genera Ruminococcus, Dorea, Clostridium and Eubacterium (A1);
at least one bacterial strain selected from the genera Phascolarctobacterium, Flavonifractor and Dialister (A8);
at least one bacterial strain selected from the genera Acetobacterium, Clostridium, Eubacterium, Moorella, Methanobrevibacter, Methanomassiliicoccus and Sporomusa (A9);
at least one bacterial strain selected from the genera Alistipes, Bacteroides, Barnesiella, Clostridium, Ruminococcus and Prevotella (A10);
at least one bacterial strain selected from the genera Clostridium, Coprococcus, Eubacterium, Flavonifractor and Flintibacter (A11);
at least one bacterial strain selected from the genera Bacteroides, Barnesiella, Bifidobacterium, Clostridium, Enterococcus, Faecalibacterium, Lactobacillus and Ruminococcus (A12);
at least one bacterial strain selected from the genera Anaerostipes, Blautia, Clostridium and Faecalibacterium (A13);
at least one bacterial strain selected from the genera Bacteroides, Bifidobacterium, Blautia, Clostridium, Faecalibacterium, Lactobacillus, Prevotella and Ruminococcus (A14); and
at least one bacterial strain selected from the genera Akkermansia, Bacteroides, Bifidobacterium and Ruminococcus (A15).

In particular the pharmaceutical or nutraceutical composition according to the invention does not comprise Blautia hydrogenotrophica.

Preferably, the pharmaceutical or nutraceutical composition comprises:
(i) Eubacterium limosum and/or Faecalibacterium prausnitzii as butyrate producers, Lactobacillus rhamnosus, Collinsella aerofaciens and/or Bifidobacterium adolescentis as lactate producers, and
Anaerotignum (former Clostridium) lactatifermentans and/or Eubacterium limosum as lactate consumers; and
(ii) at least one bacterium selected from the group consisting of Ruminococcus bromii, Phascolarctobacterium faecium and (iii) optionally Bacteroides xylanisolvens.

Preferably, the pharmaceutical or nutraceutical composition according to the invention comprises Ruminococcus bromii (A1), Faecalibacterium prausnitzii (A2), Lactobacillus rhamnosus (A3), Bifidobacterium adolescentis (A4), Anaerotignum (former Clostridium) lactatifermentans (A5), Eubacterium limosum (A6 and A9), Collinsella aerofaciens (A7) and Phascolarctobacterium faecium (A8) and optionally Bacteroides xylanisolvens (A10).

Particularly, the pharmaceutical or nutraceutical composition is free of, or essentially free of one or more of succinate, formate and lactate; and/or which further comprises propionate and/or acetate.

The pharmaceutical or nutraceutical composition according to the invention may be for use as a medicament, in particular, for use for treating a disease selected from the group consisting of cancer including gastro-intestinal cancer and colorectal cancer (CRC), intestinal infections, auto-immune disease, infections such as caused by viruses or bacteria, ulcers, gastroenteritis, Guillain-Barre syndrome, graft versus host disease (GvHD), gingivitis, nosocomial infection, Clostridium difficile infection (CDI), infection by vancomycin resistant enterococci (VRE), post-infectious diarrhea, inflammatory bowel diseases (IBD), ulcerative colitis (UC) and Crohn's disease (CD). The present invention also relates to the use of the pharmaceutical or nutraceutical composition according to the invention for the manufacture of a medicament, especially a medicament for treating a disease selected from the group consisting of cancer including gastro-intestinal cancer and colorectal cancer (CRC), intestinal infections, auto-immune disease, infections such as caused by viruses or bacteria, ulcers, gastroenteritis, Guillain-Barre syndrome, graft versus host disease (GvHD), gingivitis, nosocomial infection, Clostridium difficile infection (CDI), infection by vancomycin resistant enterococci (VRE), post-infectious diarrhea, inflammatory bowel diseases (IBD), ulcerative colitis (UC) and Crohn's disease (CD). It further relates to a method for treating a disease or disorder in a subject, the method comprising administering a therapeutically effective amount of a pharmaceutical or nutraceutical composition according to the invention. In particular, the subject has a disease selected from the group consisting of cancer including gastro-intestinal cancer and colorectal cancer (CRC), intestinal infections, auto-immune disease, infections such as caused by viruses or bacteria, ulcers, gastroenteritis, Guillain-Barre syndrome, graft versus host disease (GvHD), gingivitis, nosocomial infection, Clostridium difficile infection (CDI), infection by vancomycin resistant enterococci (VRE), post-infectious diarrhea, inflammatory bowel diseases (IBD), ulcerative colitis (UC) and Crohn's disease (CD).

In a second aspect, the invention concerns the use of glycerol to increase butyrate production of a butyrate producing bacterium in a consortium of bacteria, in particular a consortium such as disclosed herein, wherein the production of butyrate of said bacterium is increased by at least 10% in the presence of glycerol.

In a third aspect, the invention concerns a method for producing butyrate, said method comprising culturing a butyrate producing bacterium in a culture medium comprising glycerol, wherein the butyrate producing bacterium is comprised in a consortium of bacteria, in particular a consortium such as disclosed herein, and optionally recovering butyrate, preferably butyrate and bacterial cells, wherein the production of butyrate of said bacterium is increased by at least 10% in the presence of glycerol.

Particularly, glycerol is added to the culture medium at a concentration of more than 5% (v/v), preferably between 5% (v/v) and 30% (v/v) prior or during cultivation.

Preferably, butyrate, and optionally bacterial cells, are recovered when the concentration of butyrate in the culture medium is above 10 mM, preferably above 20 mM.

In a fourth aspect, the invention concerns a pharmaceutical or nutraceutical composition comprising a viable butyrate producing bacterium in a consortium of bacteria, in particular a consortium such as disclosed herein, at least 10 mM butyrate, and optionally glycerol, wherein butyrate and the butyrate producing bacterium are obtained by a method according to the invention.

In particular, such a pharmaceutical or nutraceutical composition is free of, or essentially free of one or more of succinate, formate and lactate; and/or which further comprises propionate and/or acetate.

Such a pharmaceutical or nutraceutical composition may be for use as a medicament and/or for use for treating a disease selected from the group consisting of cancer including gastro-intestinal cancer and colorectal cancer (CRC), intestinal infections, auto-immune disease, infections such as caused by viruses or bacteria, ulcers, gastroenteritis, Guillain-Barre syndrome, graft versus host disease (GvHD), gingivitis, nosocomial infection, Clostridium difficile infection (CDI), infection by vancomycin resistant enterococci (VRE), post-infectious diarrhea, inflammatory bowel diseases (IBD), ulcerative colitis (UC) and Crohn's disease (CD).

### BRIEF DESCRIPTION OF THE DRAWINGS

FIG. 1: Daily metabolite concentration of the continuously co-cultured bacterial consortium PB002 in a bioreactor before and after the supplementation of glycerol. Day of sampling from day 30 to day 55 after inoculation are represented on the x-axis and the concentrations of acetate, propionate, butyrate, formate, lactate, and succinate indicated in mM on the y-axis. Supplementation of glycerol by 30 mM at day 38 enhanced butyrate production immediately with no accumulation of the intermediate metabolites formate, lactate, or succinate.

FIG. 2: Absolute abundances of all strains of the continuously cultured consortium PB002 in a bioreactor before and after the supplementation of glycerol. Abundances were quantified using qPCR and are indicated in copies of the 16S rRNA gene/ml of culture for the strains representing A1, A2, A3, A4, A5, A6, A7, A8, and A9. Error bars represent standard deviations of technical replicates. qPCR quantification shows different abundances of the different functional groups and their stability throughout continuous fermentation. Glycerol supplementation positively affects abundance of butyrate producing strains of group A6.

FIG. 3: Relative weight change of BALB/c mice relative to their starting weight (indicated on the y-axis), over 16 days of experimentation, days are indicated on the x-axis. Mice were challenged with an acute DSS colitis adding 3% DSS in drinking water over 7 days (days 1-7) and subsequently given access to normal drinking water for the following 8 days for recovery (days 8-16). Groups represent: the control group that was not exposed to DSS, the untreated DSS group, the group treated with the composition PB002 without glycerol, the group treated with the composition PB002 containing 30% (v/v) glycerol and the group treated with human fecal microbiome transplant. Points are the means of all mice for each treatment group. Error bars indicate the SEM. Treatment group, receiving the composition PB002 with glycerol, showed a weight recovery superior to all other treatment groups.

FIG. 4: Absolute metabolite concentration of the continuously co-cultured bacterial consortium PB010 in a bioreactor at day 8 after inoculation: (1) cultured in a culture medium without glycerol, (2) cultured in a culture medium supplemented with glycerol. Absolute metabolites of the two reactors are represented on the x-axis and the concentrations of acetate, propionate, and butyrate are indicated in mM on the y-axis. There is an increase in butyrate and no accumulation of succinate, lactate or formate. The data shows that the supplementation of glycerol stimulated butyrate production of the consortium.

FIG. 5: Relative end metabolite concentration of the continuously co-cultured bacterial consortia PB002 (n=2) and PB010 (n=3) in bioreactors on day 2 and 8 of co-cultivation. All bioreactors were cultivated for 48 hours in a medium without glycerol. After day 2, glycerol was supplemented, followed by an increase in butyrate production as shown on day 8. The results show that the addition of glycerol to the growth medium leads to a reproducible and repeatable increase in butyrate production for two different consortia.

### DETAILED DESCRIPTION OF THE INVENTION

### Definitions

Unless otherwise stated, the following definitions shall apply in this specification:

As used herein, the terms “microbiome” and “microbiota” are equivalent and refer to the ecological community of commensal, symbiotic, and pathogenic microorganisms that literally share the same given habitat or host. These terms particularly refer to the human gut microbiota.

The term “Dysbiosis” is known and denotes the alteration of the microbiota in comparison to the healthy state. The microbiota state may be characterized by determining key markers, intermediate metabolites and end metabolites. Preferably, a healthy microbiota is characterized by the absence of intermediate metabolites as defined below. Accordingly, a state characterized by accumulation of intermediate metabolites is referred to as dysbiosis.

The terms “bacterium”, “bacterium strain” and “bacterial strain” can be used interchangeably and denote any bacterium of the taxonomic domain Bacteria. Due to their functions, species of the genera Methanobrevibacter and Candidatus Methanomassiliicoccus (also named herein Methanomassiliicoccus) belonging to the taxonomic domain Archaea shall be herein included in these terms. Preferably, terms “bacterium”, “bacterium strain” and “bacterial strain” refer to the taxonomic domain Bacteria.

It should be noticed that bacterium Clostridium lactatifermentans has been recently renamed Anaerotignum lactatifermentans. Then, as used herein the terms “Clostridium lactatifermentans” and “Anaerotignum lactatifermentans” have the same meaning and can be used interchangeably.

The terms “viable bacterium” and “live bacterium” can be used interchangeably and denote a bacterium which has the capacity to grow under suitable conditions. Bacterial viability can be measured using biochemical assays. Preferably, these terms relate to a bacterium strain (i) having a viability of over 50% (e.g. in pharmaceutical compositions), typically over 60% and preferably over 90%, as determined by flow cytometry.

As used herein a “butyrate producing bacterium” refers to a bacterium that is able to produce butyrate. Such a butyrate producing bacterium can naturally produce butyrate or is engineered to produce butyrate.

The term “consortium”, “microbial consortium” or “bacterial consortium” refers herein to at least two or at least three microbial organisms, preferably officiating in the same metabolic or trophic network. Preferably, microbial members of the consortium collaborate, in particular for their subsistence, into the consortium. More preferably, each bacterium of the consortium (i) produces a compound which is utilized by another bacterium of the consortium and/or (ii) utilizes a compound which is produced by another bacterium of the consortium.

As used herein, the term “inoculum” refers to a sample containing viable bacteria, intended to be introduced into an environment favorable to its multiplication, preferably a suitable culture medium, in order to produce a greater quantity of said viable bacteria or to produce a compound produced by said bacteria, for example such as butyrate. The culture is preferably conducted in an industrial scale, i.e. in particular above 200 ml, above 300 ml or above 500 ml and more preferably in a volume of at least 1 L, at least 10 L, at least 30 L, at least 50 L, at least 100 L, at least 250 L or at least 500 L.

The terms “dispersing medium”, “cultivation medium” and “culture medium” are used interchangeably herein and refer to a liquid or solid medium, preferably a liquid medium, in which one or several bacterial strains can be inoculated and/or cultivated. The composition of the culture medium depends on the nutritional requirements of the cultivated bacteria. This composition can be easily adjusted by the skilled person based on his general knowledge.

The term “short chain fatty acids” (SCFA) is also known as volatile fatty acids (VFAs) and specifically denotes fatty acids with two to six carbon atoms.

As used herein, the term “intermediate metabolite” denotes a metabolite produced by bacteria that are used as energy source or substrates by other bacteria. Such intermediate metabolites in particular may include degradation products from fibers, proteins or other organic compounds. Preferably, the intermediate metabolites are one or more of formate, lactate and succinate. More generally, the term “intermediate metabolites” may refer to an undesirable metabolite, the presence or amount of which being limited as much as possible in the final product or composition.

As used herein, the term “end metabolites” denotes the metabolites produced by bacteria that are not or only partially utilized by other bacteria. Preferably, end metabolites include butyrate, preferably butyrate and acetate and/or propionate. More generally, the term “end metabolites” may refer to a desirable metabolite, the presence or amount of which being enriched in the final product or composition.

The term “beginning of the stationary phase of growth” refers to a stage of growth that immediately follows the exponential phase of growth. It particularly refers to the phase where the exponential phase begins to decline as the available nutrients become depleted and/or inhibitory products start to accumulate. In this period, the number of living bacteria starts to remain constant in the culture.

The term “treatment” refers to any act intended to ameliorate the health status of patients or subjects such as therapy, prevention, prophylaxis and retardation of a disease. It designates both a curative treatment and/or a prophylactic treatment of a disease. A curative treatment is defined as a treatment resulting in a cure or a treatment alleviating, improving and/or eliminating, reducing and/or stabilizing the symptoms of a disease or the suffering that it causes directly or indirectly. A prophylactic treatment comprises both a treatment resulting in the prevention of a disease and a treatment reducing and/or delaying the incidence of a disease or the risk of its occurrence. In certain embodiments, such term refers to the improvement or eradication of a disease, a disorder or symptoms associated with it. The term “treatment” includes the prevention of diseases described herein and the delay of progression of diseases described herein.

As used herein, the term “a”, “an”, “the” and similar terms used in the context of the present invention (especially in the context of the claims) are to be construed to cover both the singular and plural unless otherwise indicated herein or clearly contradicted by the context.

As used herein, the terms “including”, “containing” and “comprising” are used herein in their open, non-limiting sense.

As used herein, the term “consist essentially of” refers to those elements required for a given embodiment. This term indicates the inclusion of any recited characteristics and permits the optional presence of elements that do not materially affect nor change the characteristics or functions of said embodiment. Preferably, in the context of a composition comprising bacteria, it refers to a composition that comprises the recited bacteria, and optionally includes other components such as prebiotics, at the exclusion of non-recited bacteria.

The term “at least one” means “one or more”. For instance, it refers to one, two, three or more.

Preferably, the manufacturing methods according to the invention are performed in vitro.

### Method and Use

As illustrated in the experimental section, the inventors found that butyrate producing bacteria increase their production of butyrate in the presence of glycerol.

Accordingly, in a first aspect, the invention relates to the use of glycerol to increase butyrate production of a butyrate producing bacterium, in particular in a consortium such as disclosed herein. The invention also relates to a method for producing butyrate, said method comprising culturing a butyrate producing bacterium, in particular in a consortium such as disclosed herein, in a culture medium comprising glycerol, and optionally recovering butyrate. The invention further relates to the use of glycerol and of a butyrate producing bacterium for the production of butyrate. The invention also relates to the use of a butyrate producing bacterium for producing butyrate in the presence of glycerol. The invention also relates to a manufacturing process for the production of butyrate, wherein the process utilizes glycerol for enhancing butyrate production by a butyrate producing bacteria, in particular in a consortium such as disclosed herein.

Preferably the production of butyrate of the butyrate producing bacterium is increased by at least 10%, at least 20%, at least 25%, at least 30%, at least 35%, at least 40%, at least 45%, at least 50%, at least 55%, at least 60%, at least 65% or at least 70% in the presence of glycerol. This increase is calculated by comparison with the production of butyrate of said bacterium in the same culture conditions but in the absence of glycerol. Preferably, the butyrate producing bacterium increases butyrate production by at least 20%, even more preferably by at least 40% in the presence of glycerol.

Glycerol can be added to the culture medium once or several times, at different stages of the culture, e.g. at the beginning, during the exponential phase of growth and/or during the exponential phase of growth.

Glycerol can be added to the inoculum used to seed the culture medium and/or can be added directly to the culture medium.

In an embodiment, glycerol is present in the inoculum comprising the butyrate producing bacterium. Preferably, in this embodiment, glycerol is present in the inoculum at a concentration of at least 10%, at least 20%, at least 30%, at least 40%, at least 45%, at least 50% or at least 55% (v/v).

In particular, such inoculum may be cryopreserved before use. For example, such inoculum may be obtained using any techniques known in the art, in particular by mixing bacteria with glycerol, preferably obtaining a 1:1 (v/v) mixture of bacteria and glycerol; and shock freezing with liquid nitrogen or gradually freezing to a storage temperature of at least −20° C.

Additionally or alternatively, glycerol can be added to the culture medium.

Preferably, during at least one period of the culture, glycerol is present in the culture medium at a concentration of at least 5% (v/v), preferably at a concentration between 5% and 30% (v/v). In a particular embodiment, during at least one period of the culture, glycerol is present in the culture medium at a concentration between 5% and 15% (v/v).

In particular, glycerol is still present in the culture medium at a concentration of at least 5% (v/v), preferably at a concentration between 5% and 30% (v/v) at the latest stage of the culture process. Particularly, when the method comprises a recovering step, glycerol is present at a concentration of at least 5% (v/v), preferably at a concentration between 5% and 30% (v/v) in the composition recovered.

In a particular embodiment, the method comprises culturing a butyrate producing bacteria in an appropriate culture medium comprising glycerol, and recovering butyrate, preferably butyrate and/or bacteria from the culture medium, wherein the production of butyrate by butyrate producing bacteria is increased by at least 10%, at least 20%, at least 30% or at least 40% in the presence of glycerol.

The culture medium may comprise glycerol as sole source of carbon or may comprise at least one other source such as glucose, galactose, maltose, lactose, sucrose, fructose or cellobiose, preferably glucose.

Typically, culture media include at least one carbon source (glycerol, glucose, galactose, maltose, lactose, sucrose, fructose, cellobiose), fibers (preferably pectin, arabinogalactan, beta-glucan, soluble starch, resistant starch, fructo-oligosaccharides, galacto-oligosaccharides, xylan, arabinoxylans, cellulose), proteins (preferably yeast extract, casein, skimmed milk, peptone), co-factors (short chain fatty acids, hemin, FeSO4), vitamins (preferably biotin, cobalamin, 4-aminobenzoic acid, folic acid, pyridoxamine hydrochloride), minerals (preferably sodium bicarbonate, potassium phosphate dibasic, potassium phosphate monobasic, sodium chloride, ammonium sulfate, magnesium sulfate, calcium chloride) and reducing agents (preferably cysteine, titanium(Ill)-citrate, yeast extract, sodium thioglycolate, dithiothreitol, sodium sulphide, hydrogen sulphite, ascorbate). Culture media can also comprise compound(s) preventing microbial contamination, e.g. antibiotics selected to be inefficient on desired bacteria.

In one embodiment, the culture medium further comprises intermediate metabolites, preferably one or more of formate, lactate and/or succinate.

The skilled person can easily adjust the culture medium to the nutritional requirements of the bacteria to be cultivated.

A broad range of solid or liquid culture media are known and may be used in the context of the present invention.

Suitable media include liquid media and solid supports. Liquid media generally comprise water and may thus also be termed aqueous media. Solid media may comprise a polymeric support such as agar. Preferably, the culture medium is a liquid culture medium.

During cultivation, the concentration of butyrate and/or the viability of bacteria may be measured.

The concentration of butyrate can be measured by any techniques known in the art for example by refractive index detection high pressure liquid chromatography (HPLC-RI; for example, as provided by Thermo Scientific Accela™).

The viability of bacteria can be assessed by any method known by the skilled person, for example using biochemical assays.

Optionally, the method of the invention comprises recovering butyrate, and optionally viable bacterial cells. Optionally, the method of the invention comprises recovering viable bacterial cells, in particular a consortium such as disclosed herein. Preferably, the method comprises recovering butyrate and bacterial cells, in particular a consortium such as disclosed herein. The method may also comprise recovering butyrate, glycerol and bacterial cells.

Preferably, butyrate, and optionally bacterial cells, are recovered when the concentration of butyrate in the culture medium is above 10, above 15, above 16, above 17, above 18, above 19, above 20, above 21, above 22, above 23, above 24 or above 25 mM of butyrate. More preferably, butyrate, and optionally bacterial cells, are recovered when the concentration of butyrate in the culture medium is above 20 mM.

Optionally, the concentration of one or more intermediate metabolites may be also measured.

Preferably, butyrate, and optionally bacterial cells, are recovered when the concentration of:
succinate is below 15, 10 or 5 mM, preferably below 2 mM, more preferably below 1 mM;
formate is below 15, 10 or 5 mM, preferably below 2 mM, more preferably below 1 mM; and/or
lactate is below 15, 10 or 5 mM, preferably below 2 mM, more preferably below 1 mM.

Preferably, succinate, formate and lactate present in the composition are the one produced by the bacteria of the composition, particularly when the butyrate producing bacteria is comprised in a consortium such as disclosed here below. This means that, in preferred embodiments, the method does not comprise any addition of exogenous source of intermediate metabolites before, during or after the cultivation process.

Preferably, intermediate metabolites are degraded or converted by the bacteria of the consortium. This means that, in preferred embodiments, the method does not comprise any step of depletion intermediate metabolites during or after the cultivation process.

In some embodiments, butyrate and bacterial cells are harvested during the late exponential phase of growth or at the beginning of the stationary phase of growth of the bacterial cells, especially when butyrate concentration has reached more than 10 mM, 15 mM, 20 mM or 30 mM in the culture medium.

Butyrate, and optionally bacterial cells, may be recovered when
(i) the concentration of butyrate is above 10 mM, 15 mM, 20 mM or 30 mM; and/or
(ii) the concentration of acetate is above 5 mM, 10 mM, 15 mM, 20 mM, 30 mM or 40 mM; and/or
(iii) the concentration of propionate is above 5 mM, 10 mM, 15 mM, 20 mM or 30 mM.

Preferably, butyrate, and optionally bacterial cells, are recovered when the concentration of butyrate is above 10 mM, 15 mM, 20 mM or 30 mM.

Preferably, the end metabolites, i.e. butyrate, acetate and/or propionate, are only produced by the bacteria present in the culture medium, when conducting the method of the invention for producing butyrate. This means that, in a particular embodiment, the method does not comprise any addition of exogenous source of end metabolites before, during or after the cultivation process.

In another embodiment, the method comprises, after the step of recovery butyrate, preferably butyrate and the bacterial cells, the addition of exogenous butyrate, preferably sodium butyrate and/or butyrate glyceride, in order to increase the butyrate concentration in the final product.

Glycerol can be added to the culture medium before or during the cultivation process. Preferably, glycerol is added 1, 2, 3, 4, 5, 6, 7, 8, 9, 10, 11, 12, 13 times or even continuously during the manufacturing process. It can also be added to the final product, after the step of recovery butyrate, preferably butyrate and the bacterial cells, in order to increase the glycerol concentration in the final product. Preferably, the final product comprises at least 5%, 10%, 15%, 20%, 25%, 30%, 35%, 40%, 45%, 50%, 60%, 70% or 80% (v/v) glycerol, more preferably 5-50% (v/v), preferably 10-40% (v/v), even more preferably 30% (v/v) glycerol.

In an embodiment, glycerol is added to the culture medium with the bacterial inoculum. Preferably, the inoculum comprises at least 5%, 10%, 15%, 20%, 25%, 30%, 35%, 40%, 45%, 50%, 60%, 70% or 80% (v/v) glycerol, more preferably 5-50% (v/v), preferably 10-40% (v/v), even more preferably 30% (v/v) glycerol. Depending on the glycerol concentration in the inoculum, additional amount of glycerol can be added to the culture if necessary.

Additionally or alternatively, glycerol may be added to the culture medium, i.e. from a source distinct from the inoculum, e.g. by direct addition of purified and sterile glycerol. Preferably, the culture medium comprises at least 5%, 10%, 15%, 20%, 25%, 30% (v/v) glycerol, more preferably 5-30% (v/v), preferably 10-20% (v/v), even more preferably 10% (v/v).

In one embodiment, when the method comprises recovering butyrate, optionally glycerol, and bacterial cells, in particular a consortium such as disclosed herein, the method further comprises a step of adding glycerol to the recovered composition, for example so as to adjust glycerol content to a concentration between 5% and 30% (v/v).

The butyrate producing bacterium can be cultivated using batch, fed batch or continuous fermentation.

The process of the invention may preferably be performed at a temperature suitable for bacterial growth, such as temperatures comprised between 20-40° C., and under suitable pH conditions. These conditions can be easily adjusted by the skilled person. Suitable cultivation methods are particularly disclosed in WO 2018189284 and EP18200455.6, the contents thereof being incorporated by reference.

In particular embodiments, the methods of the invention are performed in reactors. By “reactor” is meant a conventional tank or any apparatus or system for fermentation and/or bioconversion, typically selected from bioreactors, biofilters, rotary biological contactors, and other gaseous and/or liquid phase bioreactors. The apparatus which can be used according to the invention can be used continuously or in batch loads, for example for batch or fed-batch fermentation. Batch cultivation such as in an anaerobic batch or fed-batch fermentation process is known to be particularly suitable for large-scale production of bacteria.

In the reactor, to implement the method of the invention, at least one butyrate producing bacterium is used, whilst said reactor is arranged and supplied so that physicochemical conditions are set up and maintained therein so that said bacterium is operational (e.g. is able to grow and/or to produce butyrate).

Depending on the bacterium or consortium used, the method may be conducted under aerobiosis, anaerobiosis or microaerobiosis. Preferably, the method is conducted under anaerobic conditions.

The term “batch fermentation” is known and denotes a fermentation process in a bioreactor, wherein during the fermentation process no material is removed from nor added to the bioreactor. In this text, the term “batch fermentation” in particular denotes a fermentation process, wherein there is no removal of a culture suspension cultivated in the bioreactor with the exception of insignificant amounts required for analytical testing, and wherein there is no addition of fresh culture medium into the bioreactor. Furthermore, a flow of gaseous compounds into and out of the bioreactor during the fermentation process, for example inflow of inert gas to maintain anaerobic cultivating conditions or such as outflow of metabolic exhaust gas, are not considered as material added or removed from the bioreactor.

The term “fed-batch fermentation” is known and denotes a fermentation process in a bioreactor, wherein during the fermentation process no material, in particular no-culture suspension is removed from the bioreactor, except for insignificant amounts required for analytical testing and except for gaseous compounds. However, in a fed-batch fermentation process, material is added to the bioreactor during the fermentation process, in particular fresh culture medium is added. The added culture medium may be the same or different culture medium as the culture medium in the bioreactor at the beginning of the fed-batch fermentation process.

The terms “continuous culture”, “continuous cultivation” and “continuous co-cultivation” are known and refer to a cultivation of bacterial strains in a bioreactor comprising a liquid culture medium wherein during the cultivation process, materials are added and removed. In particular, the term “continuous culture” refers to a cultivation process wherein fresh medium replaces an equal volume of effluent of culture-suspension at a constant flow rate during the cultivation process.

The method according to the invention may further comprise one or more post-treatment step. The term “post treatment” preferably refers to a further processing step or downstream treatment, such as for example a preservation treatment. The post-treatment can be cryopreservation or lyophilization.

In one embodiment, the post-treatment is cryopreservation and comprises:
mixing the harvested culture-suspension with a cryoprotective solution, in particular in order to obtain a 1:1 (v/v) mixture of culture-suspension and cryopreservant, preferably glycerol, or
centrifuging the harvested culture-suspension and resuspending an obtained pellet in a mixture of the cryoprotective solution and the dispersing medium, in particular in a 1:1 (v/v) mixture of cryopreservant, preferably a mixture of glycerol and dispersing medium, and
shock freezing with liquid N2 or gradually freeze to a storage temperature of at least −20° C., in particular at −20° C. to −80° C.

In one embodiment, the post-treatment is lyophilization and comprises the steps of:
centrifuging the harvested culture-suspension and washing the obtained pellet with a buffer solution;
resuspending the pellet in a lyophilization solution and lyophilizing; and
subsequently, storing at a temperature of 4° C. or lower, or at room temperature.

In some embodiments, the method comprises the culture of only butyrate producing bacteria. In these embodiments, the culture may comprise one or several different bacterial strains.

In some other embodiments, the method comprises the culture of butyrate producing bacteria in combination with other bacteria, such as bacteria that produce lactate. In some further embodiments, the method comprises the culture of butyrate producing bacteria in a particular consortium.

Bacterial strains and consortia are more particularly defined here below.

### Bacteria and Consortia

Compositions, inoculum, uses and processes according to the invention particularly rely on butyrate producing bacteria, used alone or in combination with other bacteria, for example in a particular consortium. Such bacteria and consortia are more particularly disclosed here after.

As used herein, bacteria are defined by their capacities (for example “butyrate producers”) and are classified into functional groups. The term “functional group” as used herein, refers to functions or capacities fulfilled by bacteria. Such functions are for example capacity to degrade or convert a particular substrate, for example such as starch, and to produce a particular product or metabolite, for example such as butyrate. Generally, one bacterium is able to degrade or convert a substrate (e.g. starch) and to produce a product (e.g. butyrate). Then, a functional group comprises bacteria that are able to degrade or convert the same substrate(s) (e.g. starch) and to produce the same metabolite(s) (e.g. butyrate).

Such functions or capacities of a bacterium are well known in the art. For example, experiments are known to test if a bacterial strain belongs to a particular functional group. For example, the degradation of sugars, starches or fibers can be tested simply by providing such substrate to bacteria while observing or monitoring their growth. For example, bacteria can be characterized for growth and metabolite production on M2GSC Medium (ATCC Medium 2857) and modifications thereof whereby the carbon sources glucose, cellobiose and starch are replaced by specific substrates including intermediate metabolites and/or fibers, preferably such as found in the human intestine. The concentrations of the produced metabolites can for example be quantified by any analytic method available for the person skilled in the art such as refractive index detection high pressure liquid chromatography (HPLC-RI; for example, as provided by Thermo Scientific Accela™).

Each functional group comprises at least one bacterium of the selected bacterial strains. When bacteria are assembled into a particular consortium, each of the bacterial strain of the consortium belongs to at least one of the functional groups.

In one embodiment, the functional groups according to the invention are defined as follows:
Bacterial strains of functional group (A1), have the capacity of consuming sugars, fibers and resistant starch and producing formate and acetate.
Bacterial strains of functional group (A2) have the capacity of consuming sugars, starch and acetate and producing butyrate and formate.
Bacterial strains of functional group (A3) have the capacity of consuming sugars and to reduce oxygen, and producing lactate.
Bacterial strains of functional group (A4) have the capacity of consuming sugars, starch, and carbon dioxide, and producing lactate, formate and acetate.
Bacterial strains of functional group (A5) have the capacity of consuming protein and lactate and producing propionate and acetate.
Bacterial strains of functional group (A6) have the capacity of consuming starch, protein and lactate and producing butyrate, acetate and hydrogen.
Bacterial strains of functional group (A7) have the capacity of consuming sugar, starch and formate, and producing lactate, formate and acetate.
Bacterial strains of functional group (A8) have the capacity of consuming protein and succinate and producing propionate and acetate.
Bacterial strains of functional group (A9) have the capacity of consuming sugars, fibers, carbon dioxide, formate and hydrogen and producing acetate.
Bacterial strains of functional group (A10) have the capacity of consuming sugars, fibers, and resistant starch, and producing succinate and optionally acetate and/or propionate.
Bacterial strains of functional group (A11) have the capacity of consuming proteins and producing acetate and butyrate.
Bacterial strains of functional group (A12) have the capacity of consuming proteins, fibers, starches or sugars, and producing biogenic amines such as y-aminobutyric acid (GABA), cadaverine, dopamine, histamine, putrescine, serotonin, spermidine and/or tryptamine.
Bacterial strains of functional group (A13) have the capacity of consuming primary bile acids and producing secondary metabolites.
Bacterial strains of functional group (A14) have the capacity of producing vitamins such as cobalamin (B12), folate (B9) or riboflavin (B2).
Bacterial strains of functional group (A15) have the capacity of degrading mucus.

In pure culture, the functions of single bacteria strains (A1) to (A15) may be bidirectional. For example, (A7) may either produce or consume formate. However, when combined, the bacteria strains show the properties discussed herein, consuming intermediate metabolites (succinate, lactate, formate) and producing end metabolites (acetate, propionate, butyrate).

Preferably, bacteria disclosed herein are bacteria of the intestinal gut microbiota, in particular of human gut microbiota.

Preferably, such bacteria are not pathogenic bacteria. This means that bacteria according to the invention are known as not able to trigger any disease or disorder in a subject. Preferably, bacteria disclosed herein are bacteria strains of class I.

Preferably, the bacteria disclosed herein are facultatively or strictly anaerobic.

### Butyrate Producers

Compositions, inoculum, uses and processes according to the invention particularly rely on butyrate producing bacteria. Preferably, such butyrate producing bacteria is capable of increasing its butyrate production in the presence of glycerol. For example, such bacteria may use glycerol as a substrate, a carbon source or as an enzymatic catalyst. Particularly, said bacterial strain increases butyrate production by at least 10%, 20%, 25%, 30%, 35%, 40%, 45%, 50%, 55%, 60%, 65%, 70% in the presence of glycerol. Preferably, said bacterial strain increases butyrate production in the presence of glycerol by at least 10%, more preferable by at least 20%, even more preferably by at least 40%.

By “increased butyrate production”, “improved butyrate production” or “enhanced butyrate production” as used herein, refers to the amount or quantity of butyrate produced by a bacterium, preferably in the culture medium, that is increased in the presence of glycerol. It particularly refers to an increase of at least 10%, 20%, 25%, 30%, 35%, 40%, 45%, 50%, 55%, 60%, 65%, 70% in the presence of glycerol, compared to the production of butyrate in the same culture condition but in the absence of glycerol.

In one embodiment, the butyrate producing bacteria are bacteria from group A6, that comprises bacteria strains consuming lactate, protein and starch, producing acetate, butyrate and hydrogen. Such bacteria strains are known and include bacteria of the genera Anaerostipes, Clostridium, Coprococcus, and Eubacterium such as the species Anaerostipes caccae (e.g. DSM 14662, JCM 13470), Clostridium indolis (e.g. ATCC 25771, DSM 755, JCM 1380), Coprococcus catus (e.g. ATCC 27761), Coprococcus eutactus (e.g. ATCC 27759), Coprococcus comes (e.g. ATCC 27758), Eubacterium hallii (e.g. ATCC 27751, DSM 3353, JCM 31263), Eubacterium limosum (e.g. ATCC 8486, DSM 20543, JCM 6421), Eubacterium ramulus (e.g. ATCC 29099, DSM 15684, JCM 31355). Optionally, the butyrate producing bacteria are bacteria from group A6, that comprises bacteria strains consuming lactate, protein and starch, producing acetate, butyrate and hydrogen. Such bacteria strains are known and include bacteria of the genera Anaerostipes, Clostridium, and Eubacterium such as the species Anaerostipes caccae (e.g. DSM 14662, JCM 13470), Clostridium indolis (e.g. ATCC 25771, DSM 755, JCM 1380), Eubacterium hallii (e.g. ATCC 27751, DSM 3353, JCM 31263), Eubacterium limosum (e.g. ATCC 8486, DSM 20543, JCM 6421), Eubacterium ramulus (e.g. ATCC 29099, DSM 15684, JCM 31355).

Alternatively, or additionally butyrate producing bacteria are bacteria from group A2, that comprises bacteria strains consuming sugars, starch and acetate, producing formate and butyrate. Such bacteria strains are known and include bacteria of the genera Faecalibacterium, Roseburia, Eubacterium and Anaerostipes such as the species Faecalibacterium prausnitzii (e.g. ATCC 27768, ATCC 27766, DSM 17677, JCM 31915), Anaerostipes hadrus (e.g. ATCC 29173, DSM 3319), Roseburia spp., Roseburia intestinalis (e.g. DSM 14610, CIP 107878, JCM 31262), Roseburia hominis (e.g. DSM16839), Roseburia inulinivorans (e.g. DSM 16841, JCM 17584), Roseburia faecis (DSM 16840), Eubacterium ramulus (e.g. ATCC 29099, DSM 15684, JCM 31355) and Eubacterium rectale (e.g. DSM 17629). Optionally, butyrate producing bacteria are bacteria from group A2 include the species Faecalibacterium prausnitzii (e.g. ATCC 27768, ATCC 27766, DSM 17677, JCM 31915), Anaerostipes hadrus (e.g. ATCC 29173, DSM 3319), Roseburia intestinalis (e.g. DSM 14610, CIP 107878, JCM 31262), Eubacterium ramulus (e.g. ATCC 29099, DSM 15684, JCM 31355) and Eubacterium rectale (e.g. DSM 17629).

In a particular aspect, the bacterial strain producing butyrate is from the genus Eubacterium. Preferably, the bacterium producing butyrate is selected from the group consisting of Eubacterium hallii (e.g. ATCC 27751, DSM 3353, JCM 31263), Eubacterium limosum (e.g. ATCC 8486, DSM 20543, JCM 6421), and Eubacterium ramulus (e.g. ATCC 29099, DSM 15684). Even more preferably, the bacterial strain producing butyrate is Eubacterium limosum.

### Lactate Producers

In one embodiment, the composition, inoculum, uses and processes according to the invention comprise at least one bacterial strain producing lactate.

In one embodiment, the lactate producing bacteria are bacteria from group A3, that comprises bacteria strains consuming sugars and reducing oxygen, and producing lactate. Such bacteria strains are known and include bacteria of the genera Lactobacillus, Streptococcus, Escherichia, Lactococcus, Enterococcus such as the species Lactobacillus rhamnosus (e.g. ATCC 7469, DSM 20021, JCM 1136), Streptococcus salivarius (e.g. ATCC 7073, DSM 20560, JCM 5707), Escherichia coli (e.g. ATCC 11775, DSM 30083, JCM 1649), Lactococcus lactis (e.g. ATCC 19435, DSM 20481), Enterococcus caccae (ATCC BAA-1240, DSM 19114), and Enterococcus faecalis (e.g. ATCC 29212, DSM 2570). Optionally, the bacteria strains are selected from the species Lactobacillus rhamnosus, Streptococcus salivarius, Escherichia coli, Lactococcus lactis and Enterococcus caccae.

Alternatively, or additionally the lactate producing bacteria are bacteria from group A4, that comprises bacteria strains consuming sugars, starch, and carbon dioxide, producing lactate and formate and optionally acetate. Such bacteria strains are known and include bacteria of the genus Bifidobacterium and Roseburia, such as the species Bifidobacterium adolescentis (e.g. ATCC 15703, DSM 20083, JCM 1251), Bifidobacterium angulatum (e.g. ATCC 27535, DSM 20098), Bifidobacterium bifidum (e.g. ATCC 29521, DSM 20456, JCM 1255), Bifidobacterium breve (e.g. ATCC 1192, DSM 20213), Bifidobacterium catenulatum (e.g. ATCC 27539, DSM 16992, JCM 1194), Bifidobacterium dentium (e.g. ATCC 27534, DSM 20436, JCM 1195), Bifidobacterium gallicum (e.g. ATCC 49850, DSM 20093, JCM 8224), Bifidobacterium longum (e.g. ATCC 15707, DSM 20219, JCM 1217), Bifidobacterium pseudocatenumlatum (e.g. ATCC 27919, DSM 20438, JCM 1200) and Roseburia hominis (e.g. DSM 16839).

Alternatively, or additionally the lactate producing bacteria are bacteria from group A7, that comprises bacteria strains consuming sugar, starch and optionally formate, and producing lactate, formate and acetate. Such bacteria strains are known and include bacteria of the genus Collinsella and Roseburia, such as the species Collinsella aerofaciens (e.g. ATCC 25986, DSM 3979, JCM 10188), Collinsella intestinalis (e.g. DSM 13280, JCM 10643), Collinsella stercoris (e.g. DSM 13279, JCM 10641) and Roseburia hominis (e.g. DSM 16839).

### Lactate Consumers

In one embodiment, the composition, inoculum, uses and processes according to the invention comprise at least one bacterial strain consuming lactate.

Preferably, the lactate consuming bacteria are bacteria from group A5, that comprises bacteria strains consuming lactate and proteins, and producing propionate and acetate. Such bacteria strains are known and include bacteria of the genera Anaerotignum, Clostridium, Propionibacterium, Veillonella, Megasphaera and Coprococcus such as the species Clostridium aminovalericum (e.g. ATCC 13725, DSM 1283, JCM 1421), Clostridium celatum (e.g. ATCC 27791, DSM 1785, JCM 1394), Anaerotignum (former Clostridium) lactatifermentans (e.g. DSM 14214), Clostridium neopropionicum (e.g. DSM 3847), Clostridium propionicum (e.g. ATCC 25522, DSM 1682, JCM 1430), Megasphaera elsdenii (e.g. ATCC 25940, DSM 20460, JCM 1772), Veillonella montpellierensis (e.g. DSM 17217), Veillonella ratti (e.g. ATCC 17746, DSM 20736, JCM 6512) and Coprococcus catus (e.g. ATCC27761).

Alternatively, or additionally the lactate consuming bacteria are bacteria from group A6, that comprises bacteria strains consuming lactate, protein and starch, producing acetate, butyrate and hydrogen. Such bacteria strains are known and include bacteria of the genera Anaerostipes, Clostridium, and Eubacterium such as the species Anaerostipes caccae (e.g. DSM 14662, JCM 13470), Clostridium indolis (e.g. ATCC 25771, DSM 755, JCM 1380), Eubacterium hallii (e.g. ATCC 27751, DSM 3353, JCM 31263), Eubacterium limosum (e.g. ATCC 8486, DSM 20543, JCM 6421), Eubacterium ramulus (e.g. ATCC 29099, DSM 15684, JCM 31355).

In a particular aspect, the bacterial strain consuming lactate is from the genus Eubacterium. Preferably, the bacterium consuming lactate is selected from the group consisting of Eubacterium hallii (e.g. ATCC 27751, DSM 3353, JCM 31263), Eubacterium limosum (e.g. ATCC 8486, DSM 20543, JCM 6421), and Eubacterium ramulus (e.g. ATCC 29099, DSM 15684). Even more preferably, the bacterial strain consuming lactate is Eubacterium limosum.

Depending on the consortium, the bacterial strain consuming lactate and the strain producing butyrate can be the same or different bacterial strain. For example, bacterial strain from the genus Eubacterium, particularly Eubacterium hallii, Eubacterium limosum and Eubacterium ramulus are lactate consumers and butyrate producers. Preferably, the bacterial strain consuming lactate and producing butyrate is Eubacterium limosum.

### Additional or Optional Bacteria

In one embodiment, the composition, inoculum, uses and processes according to the invention comprise additional or optional bacteria strains.

In one embodiment, such additional or optional bacteria strains are bacteria from group A1, that comprises bacteria strains consuming sugars, fibers, and resistant starch, producing formate and acetate. Such bacteria strains are known and include bacteria of the genera Ruminococcus, Clostridium, Dorea and Eubacterium, such as the species Ruminococcus bromii (e.g. ATCC 27255, ATCC 51896), Ruminococcus lactaris (e.g. ATCC 29176), Ruminococcus champanellensis (e.g. DSM 18848, JCM 17042), Ruminococcus callidus (e.g. ATCC 27760), Ruminococcus gnavus (e.g. ATCC 29149, ATCC 35913, JCM 6515), Ruminococcus obeum (e.g. ATCC 29174, DSM 25238, JCM 31340), Dorea longicatena (e.g. DSM 13814, JCM 11232), Dorea formicigenerans (e.g. ATCC 27755, DSM 3992, JCM 31256), Clostridium scindens (e.g. DSM 5676, ATCC 35704) and Eubacterium eligens (e.g. ATCC 27750, DSM 3376).

Additionally or alternatively, such additional or optional bacteria strains are bacteria from group A8, that comprises bacteria strains consuming protein and succinate, producing propionate and acetate. Such bacteria strains are known and include bacteria of the genera Phascolarctobacterium, Dialister and Flavonifractor such as the species Phascolarctobacterium faecium (e.g. DSM 14760), Dialister succinatiphilus (e.g. DSM 21274, JCM 15077), Dialister propionifaciens (e.g. JCM 17568) and Flavonifractor plautii (e.g. ATCC 29863, DSM 4000).

Alternatively, or additionally such additional or optional bacteria strains are bacteria from group A9, that comprises bacteria strains consuming sugars, fibers, formate, carbon dioxide and hydrogen, producing acetate. Such bacteria strains are known and include bacteria of the genus Acetobacterium, Blautia, Clostridium, Moorella, Sporomusa and Eubacterium and archaea of the genera Methanobrevibacter, Methanomassiliicoccus such as the species Acetobacterium carbinolicum (e.g. ATCC BAA-990, DSM 2925), Acetobacterium malicum (e.g. DSM 4132), Acetobacterium wieringae (e.g. ATCC 43740, DSM 1911, JCM 2380), Blautia hydrogenotrophica (e.g. DSM 10507, JCM 14656), Blautia producta (e.g. ATCC 27340, DSM 2950, JCM 1471), Clostridium aceticum (e.g. ATCC35044, DSM 1496, JCM 15732), Clostridium glycolicum (e.g. ATCC 14880, DSM 1288, JCM 1401), Clostridium magnum (e.g. ATCC 49199, DSM 2767), Clostridium mayombe (e.g. ATCC 51428, DSM 2767), Methanobrevibacter smithii (e.g. ATCC 35061, DSM 861, JCM 328), Candidatus Methanomassiliicoccus intestinalis, Eubacterium hallii (e.g. ATCC 27751, DSM 3353, JCM 31263), Eubacterium limosum (e.g. ATCC 8486, DSM 20543, JCM 6421), and Eubacterium ramulus (e.g. ATCC 29099, DSM 15684, JCM).

Additionally or alternatively, such additional or optional bacteria strains are bacteria from group A10, that comprises bacteria strains consuming sugars, fibers, and resistant starch, producing succinate and optionally acetate and/or propionate. Such bacteria strains are known and include bacteria of the genera Alistipes, Bacteroides, Barnesiella, Ruminococcus and Prevotella, such as the species Bacteroides faecis (e.g. DSM 24798, JCM 16478), Bacteroides fragilis (e.g. ATCC 25285, DSM 2151, JCM 11019), Bacteroides ovatus (e.g. ATCC 8483, DSM 1896, JCM 5824), Bacteroides plebeius (e.g. DSM 17135, JCM 12973), Bacteroides uniformis (e.g. ATCC 8492, DSM 6597, JCM 5828), Bacteroides thetaiotaomicron (e.g. ATCC 29148, DSM 2079, JCM 5827), Bacteroides vulgatus (e.g. ATCC 8482, DSM 1447, JCM 5826), Bacteroides xylanisolvens (e.g. DSM 18836, JCM 15633), Barnesiella intestinihominis (e.g. DSM 21032, JCM 15079), Barnesiella viscericola (e.g. DSM 18177, JCM 13660) Ruminococcus callidus (e.g. ATCC 27760), Ruminococcus flavefaciens (e.g. DSM 25089), Prevotella copri (e.g. DSM 18205, JCM 13464), Prevotella stercorea (e.g. DSM 18206, JCM 13469), Alistipes finegoldii (e.g. DSM 1724, JCM 16770), Alistipes onderdonkii (e.g. ATCC BAA-1178, DSM 19147, JCM 16771), and Alistipes shahii (e.g. ATCC BAA-1179, DSM 19121, JCM 16773).

Additionally or alternatively, such additional or optional bacteria strains are bacteria from group A11, that comprises bacteria strains consuming proteins and producing acetate and/or butyrate. Such bacteria strains are known and include bacteria of the genera Clostridium, Coprococcus, Eubacterium, Flavonifractor and Flintibacter, such as the species Clostridium butyricum (e.g. ATCC 19398, DSM 10702, JCM 1391), Coprococcus eutactus (e.g. ATCC 27759), Eubacterium hallii (e.g. ATCC 27751, DSM 3353, JCM 31263), Flavonifractor plautii (e.g. ATCC 29863, DSM 4000) and Flintibacter butyricum (e.g. DSM 27579).

Additionally or alternatively, such additional or optional bacteria strains are bacteria from group A12, that comprise bacteria strains consuming proteins, fibers, starches or sugars producing biogenic amines such as y-aminobutyric acid (GABA), cadaverine, dopamine, histamine, putrescine, serotonin, spermidine and/or tryptamine. Such bacteria strains are known and include bacteria of the genera Bacteroides, Barnesiella, Bifidobacterium, Clostridium (only tryptamine producers), Enterococcus, Faecalibacterium, Lactobacillus and Ruminococcus (only tryptamine producers), such as the species Bacteroides caccae (e.g. DSM 19024, ATCC 43185, JCM 9498), Bacteroides faecis (e.g. DSM 24798, JCM 16478), Bacteroides fragilis (e.g. DSM 2151, ATCC 25285, JCM 11019), Bacteroides massiliensis (e.g. DSM17679), Bacteroides ovatus (e.g. DSM 1896, ATCC 8483, JCM 5824), Bacteroides uniformis (e.g. DSM 6597, ATCC 8492, JCM 5828), Bacteroides vulgatus (e.g. DSM 1447, ATCC 8482), Barnesiella intestinihominis (e.g. DSM21032), Bifidobacterium adolescentis (e.g. DSM 20083, ATCC 15703) and Lactobacillus plantarum (e.g. DSM 2601, ATCC 10241) as GABA producers, Clostridium sporogenes (e.g. ATCC 15579), Lactobacillus bulgaricus-52 (NDRI) and Ruminococcus gnavus (e.g. ATCC 29149) as tryptamine producers, Acidaminococcus intestini (e.g. DSM 21505), Bacteroides massiliensis (e.g. DSM 17679), Bacteroides stercoris (e.g. ATCC 43183), Enterococcus faecalis (ATCC 29212, DSM 2570), Enterococcus faecium (ATCC BAA-2317, DSM 7135) and Faecalibacterium prausnitzii (e.g. DSM 17677) as putrescine producers, and Clostridium bolteae (e.g. ATCC BAA-613) as spermidine producers.

Additionally or alternatively, such additional or optional bacteria strains are bacteria from group A13, that comprises bacteria strains consuming primary bile acids and producing secondary metabolites. Such bacteria strains are known and include bacteria of the genera Anaerostipes, Blautia, Clostridium and Faecalibacterium, such as the species Anaerostipes caccae (e.g. DSM14662), Blautia hydrogenotrophica (e.g. DSM 10507, JCM 14656), Clostridium bolteae (e.g. ATCC BAA-613), Clostridium scindens (e.g. DSM 5676, ATCC 35704), Clostridium symbiosum (e.g. ATCC14940) and Faecalibacterium prausnitzii (e.g. DSM 17677).

Additionally or alternatively, such additional or optional bacteria strains are bacteria from group A14, that comprise bacteria strains producing vitamins such as cobalamin (B12), folate (B9) or riboflavin (B2). Such bacteria are known in the art and include bacteria of the genera Bacteroides, Bifidobacterium, Blautia, Clostridium, Faecalibacterium, Lactobacillus, Prevotella and Ruminococcus, such as the species Bacteroides fragilis (e.g. DSM 2151, ATCC 25285, JCM 11019), Bifidobacterium adolescentis (e.g. DSM 20083, ATCC 15703), Bifidobacterium pseudocatenulatum (e.g. ATCC 27919, DSM 20438, JCM 1200), Blautia hydrogenotrophica (e.g. DSM 10507, JCM 14656), Clostridium bolteae (e.g. ATCC BAA-613), Faecalibacterium prausnitzii (e.g. DSM 17677), Lactobacillus plantarum (e.g. DSM 2601, ATCC 10241), Prevotella copri (e.g. DSM 18205, JCM 13464) and Ruminococcus lactaris (e.g. ATCC 29176).

Additionally or alternatively, such additional or optional bacteria strains are bacteria from group A15, that comprise bacteria strains consuming mucus. Such bacteria are known in the art and include bacteria of the genera Akkermansia, Bacteroides, Bifidobacterium and Ruminococcus; such as the species Akkermansia muciniphila (e.g. ATCC BAA-835), Bacteroides fragilis (e.g. DSM 2151, ATCC 25285, JCM 11019), Bacteroides thetaiotaomicron (e.g. ATCC 29148, DSM 2079, JCM 5827), Bifidobacterium bifidum (e.g. ATCC29521, DSM20456, JCM1255), Ruminococcus gnavus (e.g. ATCC29149, ATCC35913, JCM 6515) and Ruminococcus torques (e.g. ATCC 27756).

### Particular Consortium

A particular consortium according to the invention comprises at least one butyrate producing bacterial strain. Such consortium comprises 3 or 4 or 5 or more than 5 different bacterial strains. Preferably, such consortium comprises no more than 10, 15, 20 or 50 bacterial strains, even more preferably no more than 10 bacterial strains.

In particular, such consortium are designed such as to limit the accumulation of intermediate metabolites. Assembly of such consortium are particularly defined in WO 2018189284 and EP18200455.6, the content thereof being incorporated by reference.

In one embodiment, the consortium comprises or essentially consists of:
(i) at least one butyrate producing bacterium;
(ii) at least one lactate producing bacterium; and
(iii) at least one lactate consuming bacterium.

In one embodiment, the consortium comprises or essentially consists of:
(i) at least one butyrate producing bacterium selected from the genera Anaerostipes, Clostridium, Eubacterium, Coprococcus, Faecalibacterium, and Roseburia; and
at least one lactate producing bacterium selected from the genera Lactobacillus, Streptococcus, Escherichia, Lactococcus, Enterococcus, Bifidobacterium, Collinsella, and Roseburia;
at least one lactate consuming bacterium selected from the genera Anaerostipes, Eubacterium, Clostridium, Propionibacterium, Veillonella, Coprococcus and Megasphaera; and
(ii) optionally:
at least one bacterial strain selected from the genera Ruminococcus, Dorea, Clostridium and Eubacterium (A1);
at least one bacterial strain selected from the genera Phascolarctobacterium, Flavonifractor and Dialister (A8);
at least one bacterial strain selected from the genera Acetobacterium, Blautia, Clostridium, Eubacterium, Moorella, Methanobrevibacter, Methanomassiliicoccus and Sporomusa (A9);
at least one bacterial strain selected from the genera Alistipes, Bacteroides, Blautia, Barnesiella, Clostridium, Ruminococcus and Prevotella (A10);
at least one bacterial strain selected from the genera Clostridium, Coprococcus, Eubacterium, Flavonifractor and Flintibacter (A11);
at least one bacterial strain selected from the genera Bacteroides, Barnesiella, Bifidobacterium, Clostridium, Enterococcus, Faecalibacterium, Lactobacillus and Ruminococcus (A12);
at least one bacterial strain selected from the genera Anaerostipes, Blautia, Clostridium and Faecalibacterium (A13);
at least one bacterial strain selected from the genera Bacteroides, Bifidobacterium, Blautia, Clostridium, Faecalibacterium, Lactobacillus, Prevotella and Ruminococcus (A14); and
at least one bacterial strain selected from the genera Akkermansia, Bacteroides, Bifidobacterium and Ruminococcus (A15).

Optionally, the at least one butyrate producing bacterium is selected from the genera Anaerostipes, Clostridium, Eubacterium, Faecalibacterium, and Roseburia.

More specifically, the consortium comprises or essentially consists of:
(i) at least one butyrate producing bacterium selected from the genera Anaerostipes, Clostridium, Eubacterium, Coprococcus, Faecalibacterium, and Roseburia; and at least one lactate producing bacterium selected from the group consisting of Lactobacillus rhamnosus, Streptococcus salivarius, Escherichia coli, Lactococcus lactis, Enterococcus faecalis, Enterococcus caccae, Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Collinsella aerofaciens, Collinsella intestinalis, Collinsella stercoris, Roseburia hominis; and at least one lactate consuming bacterium selected from the genera Anaerostipes caccae, Clostridium indolis, Eubacterium hallii, Eubacterium limosum, Eubacterium ramulus, Clostridium aminovalericum, Clostridium celatum, Anaerotignum (former Clostridium) lactatifermentans, Clostridium neopropionicum, Clostridium propionicum, Megasphaera elsdenii, Veillonella montpellierensis, Coprococcus catus, Veillonella ratti; and
(ii) optionally:
at least one bacterial strain selected from Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens (A1);
at least one bacterial strain selected from Phascolarctobacterium faecium, Dialister succinatiphilus, Flavonifractor plautii, Dialister propionifaciens (A8);
at least one bacterial strain selected from Acetobacterium carbinolicum, Acetobacterium malicum, Acetobacterium wieringae, Blautia hydrogenotrophica, Blautia producta, Eubacterium limosum, Eubacterium hallii, Eubacterium ramulus, Clostridium aceticum, Clostridium glycolicum, Clostridium magnum, Clostridium mayombe, Methanobrevibacter smithii, Candidatus Methanomassiliicoccus intestinalis (A9);
at least one bacterial strain selected from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii (A10);
at least one bacterial strain selected from Clostridium butyricum, Coprococcus eutactus, Eubacterium hallii, Flavonifractor plautii and Flintibacter butyricum (A11);
at least one bacterial strain selected from Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris, Enterococcus faecalis, Enterococcus faecium and Faecalibacterium prausnitzii as putrescine producers, and Clostridium bolteae (A12);
at least one bacterial strain selected from Anaerostipes caccae, Blautia hydrogenotrophica, Clostridium bolteae, Clostridium scindens, Clostridium symbiosum and Faecalibacterium prausnitzii (A13);
at least one bacterial strain selected from Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris (A14); and
at least one bacterial strain selected from Akkermansia muciniphila, Bacteroides fragilis, Bacteroides thetaiotaomicron, Bifidobacterium bifidum, Ruminococcus gnavus and Ruminococcus torques (A15).

Optionally, the at least one butyrate producing bacterium is selected from the group consisting of Faecalibacterium prausnitzii, Anaerostipes hadrus, Roseburia intestinalis, Eubacterium ramulus, Eubacterium rectale, Anaerostipes caccae, Clostridium indolis, Eubacterium hallii, and Eubacterium limosum.

In one embodiment, the consortium comprises or essentially consists of butyrate producing bacteria as disclosed herein, especially Eubacterium limosum, and:
(i) at least one lactate producing bacterium selected from the genera Lactobacillus, Streptococcus, Escherichia, Lactococcus, Enterococcus, Bifidobacterium, Collinsella, and Roseburia; and
at least one lactate consuming bacterium selected from the genera Anaerostipes, Eubacterium, Clostridium, Propionibacterium, Veillonella, Coprococcus and Megasphaera; and
(ii) optionally:
at least one bacterial strain selected from the genera Ruminococcus, Dorea, Clostridium and Eubacterium (A1);
at least one bacterial strain selected from the genera Phascolarctobacterium, Flavonifractor and Dialister (A8);
at least one bacterial strain selected from the genera Acetobacterium, Blautia, Clostridium, Eubacterium, Moorella, Methanobrevibacter, Methanomassiliicoccus and Sporomusa (A9);
at least one bacterial strain selected from the genera Alistipes, Bacteroides, Blautia, Barnesiella, Clostridium, Ruminococcus and Prevotella (A10);
at least one bacterial strain selected from the genera Clostridium, Coprococcus, Eubacterium, Flavonifractor and Flintibacter (A11);
at least one bacterial strain selected from the genera Bacteroides, Barnesiella, Bifidobacterium, Clostridium, Enterococcus, Faecalibacterium, Lactobacillus and Ruminococcus (A12);
at least one bacterial strain selected from the genera Anaerostipes, Blautia, Clostridium and Faecalibacterium (A13);
at least one bacterial strain selected from the genera Bacteroides, Bifidobacterium, Blautia, Clostridium, Faecalibacterium, Lactobacillus, Prevotella and Ruminococcus (A14); and
at least one bacterial strain selected from the genera Akkermansia, Bacteroides, Bifidobacterium and Ruminococcus (A15).

More specifically, the consortium comprises or essentially consists of butyrate producing bacteria as disclosed herein, especially Eubacterium limosum, and:
(i) at least one lactate producing bacterium selected from the group consisting of Lactobacillus rhamnosus, Streptococcus salivarius, Escherichia coli, Lactococcus lactis, Enterococcus faecalis, Enterococcus caccae, Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Collinsella aerofaciens, Collinsella intestinalis, Collinsella stercoris, Roseburia hominis; and
at least one lactate consuming bacterium selected from the genera Anaerostipes caccae, Clostridium indolis, Eubacterium hallii, Eubacterium limosum, Eubacterium ramulus, Clostridium aminovalericum, Clostridium celatum, Anaerotignum (former Clostridium) lactatifermentans, Clostridium neopropionicum, Clostridium propionicum, Megasphaera elsdenii, Veillonella montpellierensis, Coprococcus catus, Veillonella ratti; and
(ii) optionally:
at least one bacterial strain selected from Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens (A1);
at least one bacterial strain selected from Phascolarctobacterium faecium, Dialister succinatiphilus, Flavonifractor plautii, Dialister propionifaciens (A8);
at least one bacterial strain selected from Acetobacterium carbinolicum, Acetobacterium malicum, Acetobacterium wieringae, Blautia hydrogenotrophica, Blautia producta, Eubacterium limosum, Eubacterium hallii, Eubacterium ramulus, Clostridium aceticum, Clostridium glycolicum, Clostridium magnum, Clostridium mayombe, Methanobrevibacter smithii, Candidatus Methanomassiliicoccus intestinalis (A9);
at least one bacterial strain selected from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii (A10);
at least one bacterial strain selected from Clostridium butyricum, Coprococcus eutactus, Eubacterium hallii, Flavonifractor plautii and Flintibacter butyricum (A11);
at least one bacterial strain selected from Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris, Enterococcus faecalis, Enterococcus faecium and Faecalibacterium prausnitzii as putrescine producers, and Clostridium bolteae (A12);
at least one bacterial strain selected from Anaerostipes caccae, Blautia hydrogenotrophica, Clostridium bolteae, Clostridium scindens, Clostridium symbiosum and Faecalibacterium prausnitzii (A13);
at least one bacterial strain selected from Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris (A14); and
at least one bacterial strain selected from Akkermansia muciniphila, Bacteroides fragilis, Bacteroides thetaiotaomicron, Bifidobacterium bifidum, Ruminococcus gnavus and Ruminococcus torques (A15).

In a particular embodiment, the consortium comprises or consists essentially of:
(i) Eubacterium limosum and/or Faecalibacterium prausnitzii as butyrate producers,
Lactobacillus rhamnosus, Collinsella aerofaciens and/or Bifidobacterium adolescentis as lactate producers, and
Anaerotignum (former Clostridium) lactatifermentans and/or Eubacterium limosum as lactate consumers; and
(ii) at least one bacterium selected from the group consisting of Ruminococcus bromii, Phascolarctobacterium faecium, and
(iii) optionally Bacteroides xylanisolvens.

Preferably the consortium comprises or consists essentially of Ruminococcus bromii (A1), Faecalibacterium prausnitzii (A2), Lactobacillus rhamnosus (A3), Bifidobacterium adolescentis (A4), Anaerotignum (former Clostridium) lactatifermentans (A5), Eubacterium limosum (A6 and A9), Collinsella aerofaciens (A7) and Phascolarctobacterium faecium (A8) and optionally Bacteroides xylanisolvens (A10).

In one embodiment, the consortium comprises or essentially consists of Ruminococcus bromii (A1), Faecalibacterium prausnitzii (A2), Lactobacillus rhamnosus (A3), Bifidobacterium adolescentis (A4), Anaerotignum (former Clostridium) lactatifermentans (A5), Eubacterium limosum (A6), Collinsella aerofaciens (A7), Phascolarctobacterium faecium (A8), and Blautia hydrogenotrophica (A9) and optionally Bacteroides xylanisolvens (A10).

Alternatively, the consortium comprises or essentially consists of Eubacterium eligens (A1), Roseburia intestinalis (A2), Enterococcus faecalis (A3), Roseburia hominis (A4 and A7), Coprococcus catus (A5), Eubacterium hallii (A6), Flavonifractor plautii (A8), Eubacterium limosum (A9) and optionally Bacteroides xylanisolvens (A10).

Alternatively, the consortium comprises or essentially consists of (A1), Roseburia intestinalis (A2), Enterococcus faecalis (A3), Roseburia hominis (A4 and A7), Coprococcus catus (A5), Eubacterium limosum (A6 and A9), and Flavonifractor plautii (A8).

In a particular aspect, preferably when the consortium comprises an Eubacterium as a butyrate producing bacteria, preferably Eubacterium limosum, the consortium is such that it does not comprise Blautia hydrogenotrophica.

In another particular aspect, the consortium is such that it does not comprise a bacterium from the genus Blautia, especially Blautia hydrogenotrophica and/or Blautia producta, particularly when the consortium comprises an Eubacterium as a butyrate producing bacteria, preferably Eubacterium limosum.

Additionally or alternatively, in particular when the consortium comprises a bacterium of the genera Eubacterium as a butyrate producing bacteria, preferably Eubacterium limosum, the consortium is such that it does not comprise:
an archaea of the genus Methanobrevibacter or Methanomassiliicoccus, preferably Methanobrevibacter smithii and/or Candidatus Methanomassiliicoccus intestinalis;
a bacterium of the genera Acetobacterium, preferably Acetobacterium carbinolicum, Acetobacterium malicum and/or Acetobacterium wieringae;
a bacterium of the genera Moorella and/or Sporomusa; and/or
a bacterium selected from Clostridium aceticum, Clostridium magnum, Clostridium glycolicum and/or Clostridium mayombe.

Then, in one embodiment, the consortium comprises or essentially consists of butyrate producing bacteria as disclosed herein, especially Eubacterium limosum, and:
(i) at least one lactate producing bacterium selected from the genera Lactobacillus, Streptococcus, Escherichia, Lactococcus, Enterococcus, Bifidobacterium, Collinsella, and Roseburia; and
at least one lactate consuming bacterium selected from the genera Anaerostipes, Eubacterium, Clostridium, Anaerotignum, Propionibacterium, Veillonella, Coprococcus and Megasphaera; and
(ii) optionally:
at least one bacterial strain selected from the genera Ruminococcus, Dorea, Clostridium and Eubacterium (A1);
at least one bacterial strain selected from the genera Phascolarctobacterium, Flavonifractor and Dialister (A8);
at least one bacterial strain selected from the genera Alistipes, Bacteroides, Barnesiella, Clostridium, Ruminococcus and Prevotella (A10);
at least one bacterial strain selected from the genera Clostridium, Coprococcus, Eubacterium, Flavonifractor and Flintibacter (A11);
at least one bacterial strain selected from the genera Bacteroides, Barnesiella, Bifidobacterium, Clostridium, Enterococcus, Faecalibacterium, Lactobacillus and Ruminococcus (A12);
at least one bacterial strain selected from the genera Anaerostipes, Clostridium and Faecalibacterium (A13);
at least one bacterial strain selected from the genera Bacteroides, Bifidobacterium, Clostridium, Faecalibacterium, Lactobacillus, Prevotella and Ruminococcus (A14); and
at least one bacterial strain selected from the genera Akkermansia, Bacteroides, Bifidobacterium and Ruminococcus (A15).

Particularly, the consortium comprises or essentially consists of butyrate producing bacteria as disclosed herein, especially Eubacterium limosum, and:
(i) at least one lactate producing bacterium selected from the genera Lactobacillus, Streptococcus, Escherichia, Lactococcus, Enterococcus, Bifidobacterium, Collinsella, and Roseburia; and
at least one lactate consuming bacterium selected from the genera Anaerostipes, Eubacterium, Propionibacterium, Veillonella, Coprococcus and Megasphaera; and
   (ii) optionally:
at least one bacterial strain selected from the genera Ruminococcus, Dorea, and Eubacterium (A1),
at least one bacterial strain selected from the genera Phascolarctobacterium, Flavonifractor and Dialister (A8);
at least one bacterial strain selected from the genera Alistipes, Bacteroides, Barnesiella, Ruminococcus and Prevotella (A10);
at least one bacterial strain selected from the genera Coprococcus, Eubacterium, Flavonifractor and Flintibacter (A11);
at least one bacterial strain selected from the genera Bacteroides, Barnesiella, Bifidobacterium, Enterococcus, Faecalibacterium, Lactobacillus and Ruminococcus (A12);
at least one bacterial strain selected from the genera Anaerostipes and Faecalibacterium (A13);
at least one bacterial strain selected from the genera Bacteroides, Bifidobacterium, Faecalibacterium, Lactobacillus, Prevotella and Ruminococcus (A14); and
at least one bacterial strain selected from the genera Akkermansia, Bacteroides, Bifidobacterium and Ruminococcus (A15).

More specifically, the consortium comprises or essentially consists of butyrate producing bacteria as disclosed herein, especially Eubacterium limosum, and:
(i) at least one lactate producing bacterium selected from the group consisting of Lactobacillus rhamnosus, Streptococcus salivarius, Escherichia coli, Lactococcus lactis, Enterococcus faecalis, Enterococcus caccae, Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Collinsella aerofaciens, Collinsella intestinalis, Collinsella stercoris, Roseburia hominis; and
at least one lactate consuming bacterium selected from the genera Anaerostipes caccae, Clostridium indolis, Eubacterium hallii, Eubacterium limosum, Eubacterium ramulus, Clostridium aminovalericum, Clostridium celatum, Anaerotignum (former Clostridium) lactatifermentans, Clostridium neopropionicum, Clostridium propionicum, Megasphaera elsdenii, Veillonella montpellierensis, Coprococcus catus, Veillonella ratti; and
(ii) optionally:
at least one bacterial strain selected from Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens (A1);
at least one bacterial strain selected from Phascolarctobacterium faecium, Dialister succinatiphilus, Flavonifractor plautii, Dialister propionifaciens (A8);
at least one bacterial strain selected from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii (A10);
at least one bacterial strain selected from Clostridium butyricum, Coprococcus eutactus, Eubacterium hallii, Flavonifractor plautii and Flintibacter butyricum (A11);
at least one bacterial strain selected from Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris, Enterococcus faecalis, Enterococcus faecium and Faecalibacterium prausnitzii as putrescine producers, and Clostridium bolteae (A12);
at least one bacterial strain selected from Anaerostipes caccae, Clostridium bolteae, Clostridium scindens, Clostridium symbiosum and Faecalibacterium prausnitzii (A13);
at least one bacterial strain selected from Bacteroides fragilis, Bifidobacterium adolescentis, Bifidobacterium pseudocatenulatum, Clostridium bolteae, Faecalibacterium prausnitzii, Lactobacillus plantarum, Prevotella copri and Ruminococcus lactaris (A14); and
at least one bacterial strain selected from Akkermansia muciniphila, Bacteroides fragilis, Bacteroides thetaiotaomicron, Bifidobacterium bifidum, Ruminococcus gnavus and Ruminococcus torques (A15).

Particularly, the consortium comprises or essentially consists of butyrate producing bacteria as disclosed herein, especially Eubacterium limosum, and:
(i) at least one lactate producing bacterium selected from the group consisting of Lactobacillus rhamnosus, Streptococcus salivarius, Escherichia coli, Lactococcus lactis, Enterococcus faecalis, Enterococcus caccae, Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Collinsella aerofaciens, Collinsella intestinalis, Collinsella stercoris, Roseburia hominis; and
at least one lactate consuming bacterium selected from the genera Anaerostipes caccae, Eubacterium hallii, Eubacterium limosum, Eubacterium ramulus, Megasphaera elsdenii, Veillonella montpellierensis, Coprococcus catus, Veillonella ratti; and
(ii) optionally:
at least one bacterial strain selected from Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens (A1);
at least one bacterial strain selected from Phascolarctobacterium faecium, Dialister succinatiphilus, Flavonifractor plautii, Dialister propionifaciens (A8);
at least one bacterial strain selected from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii (A10);
at least one bacterial strain selected from Coprococcus eutactus, Eubacterium hallii, Flavonifractor plautii and Flintibacter butyricum (A11);
at least one bacterial strain selected from Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Lactobacillus bulgaricus-52 and Ruminococcus gnavus as tryptamine producers, Acidaminococcus intestini, Bacteroides massiliensis, Bacteroides stercoris, Enterococcus faecalis, Enterococcus faecium and Faecalibacterium prausnitzii as putrescine producers (A12);
at least one bacterial strain selected from Anaerostipes caccae, and Faecalibacterium prausnitzii (A13);
at least one bacterial strain selected from Bacteroides fragilis, Bifidobacterium adolescentis, Bifidobacterium pseudocatenulatum, Faecalibacterium prausnitzii, Lactobacillus plantarum, Prevotella copri and Ruminococcus lactaris (A14); and
at least one bacterial strain selected from Akkermansia muciniphila, Bacteroides fragilis, Bacteroides thetaiotaomicron, Bifidobacterium bifidum, Ruminococcus gnavus and Ruminococcus torques (A15).

### Compositions

In a further aspect, the present invention relates to a composition comprising at least a viable butyrate producing bacterium and butyrate, and optionally glycerol. In particular, said composition may be obtained using the method for producing butyrate of the invention.

The compositions described herein are useful as pharmaceuticals in the applications described herein. Particularly, the compositions described herein are pharmaceutical compositions. As used herein, a “pharmaceutical composition” may refer to a preparation of one or more active agents, in particular one or more bacteria, preferably a consortium, as defined herein, formulated with optional ingredients such as physiologically suitable carriers and/or excipients.

The composition described herein may also be nutraceutical or dietary compositions, in particular such as dietary or food supplement.

The term “nutraceutical” refers to a composition or product made from or comprising food substances, but made available in tablet, powder, portion or other galenic forms not usually associated with food. This definition includes dietary supplements or meal replacements.

By “food supplement”, it is meant here any composition which is formulated and administered separately from other foods and is intended to supplement the nutritional intake of a subject in a suitable form, in particular in the form of capsules, tablets, soft capsules, sachets, stick-packs, syrup, dropper, or any other adapted form well known to the person in the trade.

The terms “food”, “food product” and “foodstuff” are used interchangeably here and include, in addition to foods commonly consumed by humans, functional foods.

Preferably, the viable butyrate producing bacterium is as defined above. In particular, the butyrate production of said bacterium is increased by at least 10% in the presence of glycerol.

In preferred embodiments, the pharmaceutical composition according to the invention comprises:
a. at least one bacterial strain producing lactate;
b. at least one bacterial strain consuming lactate; and
c. at least one bacterial strain producing butyrate, the production of butyrate of said bacterium being increased by at least 10% in the presence of glycerol; and
d. at least 10 mM of butyrate.

Preferably, the bacterial strains are such as described above under the “Bacteria and consortia” paragraph.

In an embodiment, the composition comprises (a) butyrate producing bacteria as disclosed herein, especially Eubacterium limosum, and:
(b) at least one lactate producing bacterium selected from the genera Lactobacillus, Streptococcus, Escherichia, Lactococcus, Enterococcus, Bifidobacterium, Collinsella, and Roseburia; and
(c) at least one lactate consuming bacterium selected from the genera Anaerostipes, Eubacterium, Clostridium, Propionibacterium, Veillonella, Coprococcus and Megasphaera; and
optionally:
at least one bacterial strain selected from the genera Ruminococcus, Dorea, Clostridium and Eubacterium (A1);
at least one bacterial strain selected from the genera Phascolarctobacterium, Flavonifractor and Dialister (A8);
at least one bacterial strain selected from the genera Acetobacterium, Blautia, Clostridium, Eubacterium, Moorella, Methanobrevibacter, Methanomassiliicoccus and Sporomusa (A9);
at least one bacterial strain selected from the genera Alistipes, Bacteroides, Blautia, Barnesiella, Clostridium, Ruminococcus and Prevotella (A10);
at least one bacterial strain selected from the genera Clostridium, Coprococcus, Eubacterium, Flavonifractor and Flintibacter (A11);
at least one bacterial strain selected from the genera Bacteroides, Barnesiella, Bifidobacterium, Clostridium, Enterococcus, Faecalibacterium, Lactobacillus and Ruminococcus (A12);
at least one bacterial strain selected from the genera Anaerostipes, Blautia, Clostridium and Faecalibacterium (A13);
at least one bacterial strain selected from the genera Bacteroides, Bifidobacterium, Blautia, Clostridium, Faecalibacterium, Lactobacillus, Prevotella and Ruminococcus (A14); and
at least one bacterial strain selected from the genera Akkermansia, Bacteroides, Bifidobacterium and Ruminococcus (A15); and
(d) at least 10 mM butyrate, preferably at least 20 mM butyrate.

Preferably, the composition further comprises at least 5% of glycerol.

More specifically, the composition may comprise a consortium comprising or essentially consisting of (a) butyrate producing bacteria as disclosed herein, especially Eubacterium limosum, and:
(b) at least one lactate producing bacterium selected from the group consisting of Lactobacillus rhamnosus, Streptococcus salivarius, Escherichia coli, Lactococcus lactis, Enterococcus faecalis, Enterococcus caccae, Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Collinsella aerofaciens, Collinsella intestinalis, Collinsella stercoris, Roseburia hominis; and
(c) at least one lactate consuming bacterium selected from the genera Anaerostipes caccae, Clostridium indolis, Eubacterium hallii, Eubacterium limosum, Eubacterium ramulus, Clostridium aminovalericum, Clostridium celatum, Anaerotignum (former Clostridium) lactatifermentans, Clostridium neopropionicum, Clostridium propionicum, Megasphaera elsdenii, Veillonella montpellierensis, Coprococcus catus, Veillonella ratti; and
optionally:
at least one bacterial strain selected from Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens (A1);
at least one bacterial strain selected from Phascolarctobacterium faecium, Dialister succinatiphilus, Flavonifractor plautii, Dialister propionifaciens (A8);
at least one bacterial strain selected from Acetobacterium carbinolicum, Acetobacterium malicum, Acetobacterium wieringae, Blautia hydrogenotrophica, Blautia producta, Eubacterium limosum, Eubacterium hallii, Eubacterium ramulus, Clostridium aceticum, Clostridium glycolicum, Clostridium magnum, Clostridium mayombe, Methanobrevibacter smithii, Candidatus Methanomassiliicoccus intestinalis (A9);
at least one bacterial strain selected from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii (A10);
at least one bacterial strain selected from Clostridium butyricum, Coprococcus eutactus, Eubacterium hallii, Flavonifractor plautii and Flintibacter butyricum (A11);
at least one bacterial strain selected from Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestine, Bacteroides massiliensis, Bacteroides stercoris, Enterococcus faecalis, Enterococcus faecium and Faecalibacterium prausnitzii as putrescine producers, and Clostridium bolteae (A12);
at least one bacterial strain selected from Anaerostipes caccae, Blautia hydrogenotrophica, Clostridium bolteae, Clostridium scindens, Clostridium symbiosum and Faecalibacterium prausnitzii (A13);
at least one bacterial strain selected from Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris (A14); and
at least one bacterial strain selected from Akkermansia muciniphila, Bacteroides fragilis, Bacteroides thetaiotaomicron, Bifidobacterium bifidum, Ruminococcus gnavus and Ruminococcus torques (A15).
(d) at least 10 mM butyrate, preferably at least 20 mM butyrate.

Preferably, the composition further comprises at least 5% of glycerol.

In a particular embodiment, the composition comprises or consists essentially of:
a. Eubacterium limosum and/or Faecalibacterium prausnitzii as butyrate producers;
b. Lactobacillus rhamnosus, Collinsella aerofaciens and/or Bifidobacterium adolescentis as lactate producers; and
c. Anaerotignum (former Clostridium) lactatifermentans and/or Eubacterium limosum as lactate consumers; and
d. at least 10 mM of butyrate, preferably at least 20 mM butyrate; and at least one bacterium selected from the group consisting of Ruminococcus bromii, Phascolarctobacterium faecium, and optionally Bacteroides xylanisolvens.

Preferably, the composition further comprises at least 5% of glycerol.

Preferably, the composition comprises or consists essentially of Ruminococcus bromii (A1), Faecalibacterium prausnitzii (A2), Lactobacillus rhamnosus (A3), Bifidobacterium adolescentis (A4), Anaerotignum (former Clostridium) lactatifermentans (A5), Eubacterium limosum (A6 and A9), Collinsella aerofaciens (A7) and Phascolarctobacterium faecium (A8) and optionally Bacteroides xylanisolvens (A10); at least 10 mM of butyrate, preferably at least 20 mM butyrate; and optionally at least 5% of glycerol. Preferably, the composition further comprises at least 5% of glycerol.

In a particular aspect, when the composition comprises an Eubacterium as a butyrate producing bacteria, preferably Eubacterium limosum, the composition is such that it does not comprise Blautia hydrogenotrophica.

In another particular aspect, the composition is such that it does not comprise a bacterium from the genus Blautia, especially Blautia hydrogenotrophica and/or Blautia producta, when the composition comprises an Eubacterium as a butyrate producing bacteria, preferably Eubacterium limosum.

Additionally or alternatively, when the composition comprises butyrate producing bacteria as disclosed herein, especially Eubacterium, preferably Eubacterium limosum, the composition is such that it does not comprise:
an archaea of the genus Methanobrevibacter or Methanomassiliicoccus, preferably Methanobrevibacter smithii and/or Candidatus Methanomassiliicoccus intestinalis;
a bacterium of the genera Acetobacterium, preferably Acetobacterium carbinolicum, Acetobacterium malicum and/or Acetobacterium wieringae;
a bacterium of the genera Moorella and/or Sporomusa; and/or
a bacterium of the genus Clostridium, preferably Clostridium aceticum, Clostridium magnum and/or Clostridium mayombe.

In one embodiment, the composition comprises or essentially consists of Ruminococcus bromii (A1), Faecalibacterium prausnitzii (A2), Lactobacillus rhamnosus (A3), Bifidobacterium adolescentis (A4), Anaerotignum (former Clostridium) lactatifermentans (A5), Eubacterium limosum (A6), Collinsella aerofaciens (A7), Phascolarctobacterium faecium (A8), and Blautia hydrogenotrophica (A9) and optionally Bacteroides xylanisolvens (A10); and at least 10 mM of butyrate, preferably at least 20 mM butyrate. Preferably, the composition further comprises at least 5% of glycerol.

Alternatively, the composition comprises or essentially consists of Eubacterium eligens (A1), Roseburia intestinalis (A2), Enterococcus faecalis (A3), Roseburia hominis (A4 and A7), Coprococcus catus (A5), Eubacterium hallii (A6), Flavonifractor plautii (A8), Eubacterium limosum (A9) and optionally Bacteroides xylanisolvens (A10); and at least 10 mM of butyrate, preferably at least 20 mM butyrate. Preferably, the composition further comprises at least 5% of glycerol.

Alternatively, the composition comprises or essentially consists of (A1), Roseburia intestinalis (A2), Enterococcus faecalis (A3), Roseburia hominis (A4 and A7), Coprococcus catus (A5), Eubacterium limosum (A6 and A9), and Flavonifractor plautii (A8); and at least 10 mM of butyrate, preferably at least 20 mM butyrate. Preferably, the composition further comprises at least 5% of glycerol.

Preferably, the pharmaceutical composition comprises an effective therapeutic amount of bacteria, preferably 10³ to 10¹⁴ CFU (colony forming units) of bacteria per ml or mg of the pharmaceutical composition.

Preferably, the amount of butyrate in the composition is above 15 mM, above 16 mM, above 17 mM, above 18 mM, above 19 mM, above 20 mM, above 21 mM, above 22 mM, above 23 mM, above 24 mM, above 25 mM or above 30 mM. Advantageously, the amount of butyrate is below 60 mM.

Preferably, the pharmaceutical composition further comprises at least 5%, 10%, 15%, 20%, 25%, 30%, 35%, 40%, 45%, 50% (v/v) glycerol. Preferably, glycerol is of a technical or industrial grade (i.e. comprising at least 95, 96, 97, 98 or 99% glycerol).

### Advantageously:

the amount of succinate in the composition is below 15, 10 or 5 mM, preferably below 2 mM, much preferably below 1 mM; and/or
the amount of formate is below 15, 10 or 5 mM, preferably below 2 mM, much preferably below 1 mM; and/or
the amount of lactate is below 15, 10 or 5 mM, preferably below 2 mM, much preferably below 1 mM.

The concentration of these metabolites may be determined using HPLC-RI or any other method known by the skilled person.

Preferably, the composition according to the invention is free of, or essentially free of, intermediate metabolites, especially one or more of succinate, formate and lactate.

As used herein, the term “essentially free” refers to a concentration below detection limit, preferably below the detection limit of HPLC-RI technology. The detection limit of HPLC-RI technology is about 1 mM. Thus, in preferred embodiments, this term refers to a concentration below of 1 mM or less.

Additionally, the composition according to the invention may further comprises other end metabolites such as propionate and/or acetate.

Preferably, the composition according to the invention comprises at least one bacterial strain producing lactate, at least one bacterial strain consuming lactate; at least one bacterial strain producing butyrate, the production of butyrate of said bacterium being increased by at least 10% in the presence of glycerol; and at least 20 mM of butyrate, and further comprises acetate and/or propionate, preferably acetate and propionate.

Such bacterial strains are particularly disclosed under the “bacteria and consortia” paragraph.

Preferably, the composition according to the invention further comprises at least one bacterium producing acetate and/or at least one bacterium producing propionate. More preferably, the composition of the invention comprises at least one bacterium producing acetate and at least one bacterium producing propionate.

Advantageously, the composition comprises
at least 10 Mm acetate, preferably at least 15, 20, 25 or 30 mM, much preferably at least 50 mM. Advantageously, the amount of acetate is below 120 mM; and/or
at least 2 mM propionate, preferably at least 5 mM, much preferably at least 10, 15, 20, 25 or 30 mM. Advantageously, the amount of propionate is below 60 mM.

Alternatively, the butyrate used in the composition is selected from butyrate produced by the bacteria, sodium butyrate and butyrate glycerides, and mixtures thereof. Butyrate glycerides, including mono-, di- and tri-butyrin, consist of a varied number of butyric acid molecules attached to glycerol backbone.

The composition of the invention may comprise additional compounds such as other intermediate and end metabolites or other active ingredients. In particular, when the composition is obtained using the method of the invention, ingredients may vary upon the nature of bacteria used during the production and then included in the composition.

The compositions of the present invention can be in a form suitable for any conventional route of administration or use. The composition may comprise at least one pharmaceutically acceptable carrier and/or excipient. A “pharmaceutically acceptable vehicle” or “pharmaceutically acceptable carrier” as referred to herein, is any known compound or combination of compounds that are known to those skilled in the art to be useful in formulating pharmaceutical compositions. Examples of such carriers or excipients include, but are not limited to, adjuvants, diluents, binders, stabilizers, buffers, salts, lipophilic solvents, preservatives.

Such pharmaceutical and nutraceutical compositions may be formulated according to known principles and adapted to various modes of administration. The pharmaceutical compositions have to be formulated in order to preserve viability of the bacteria present in the composition. Such formulations are known by the skilled person.

In a particular embodiment, the pharmaceutical composition of the invention is adapted to rectal administration.

In another particular embodiment, the pharmaceutical or nutraceutical composition of the invention is adapted to oral administration.

For oral administration, the pharmaceutical or nutraceutical composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

Well-known thickening agents may also be added to compositions such as corn starch, agar, natural or synthetic gums, resins, methylcellulose, sodium carboxymethylcellulose, guar, xanthan and the like. Preservatives may also be included in the composition, including methylparaben, propylparaben, benzyl alcohol and ethylene diamine tetraacetate salts.

Pharmaceutical or nutraceutical compositions according to the invention may be formulated to release the active ingredients substantially immediately upon administration or at any predetermined time or time period after administration.

In one embodiment, the pharmaceutical or nutraceutical composition further comprises prebiotics. As used herein, a “prebiotic” refers to an ingredient or substrate that, selectively used by a bacterium of the host or of the composition according to the invention, confers a benefit on health of the host or on the bacterium itself. It can induce beneficial changes, both in the composition comprising the bacterium and/or activity of such bacterium. For example, prebiotic can be added in the composition according to the invention so that the bacteria of the composition are in a favorable environment upon administration. Prebiotic may also be an edible food or drink or an ingredient thereof. Prebiotics include, but are not limited to, amino acids, biotin, fructo-oligosaccharide, galacto-oligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugar beet fiber, pea fiber, corn bran, and oat fiber) and xylooligosaccharides.

For transmucosal administration, nasal sprays, rectal or vaginal suppositories can be used. The active compounds can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

In a preferred embodiment, the composition is in a gastro-resistant oral form allowing the bacteria contained in the composition to pass the stomach and be released into the intestine. Preferably, the composition is formulated using an enteric material which is stable at acid pH and labile at basic pH, which means that it does not dissolve in the stomach, but dissolves in the intestine. The material that can be used in enteric coatings includes, for example, alginic acid, cellulose acetate phthalate, plastics, waxes, shellac and fatty acids (e.g. stearic acid or palmitic acid).

The composition of the excipient or carrier can be modified as long as it does not significantly interfere with the viability of the bacteria present in the composition of the invention.

### Uses of the Compositions

In a further aspect, the present invention also relates to the use of the pharmaceutical composition as a medicament, especially in the treatment of a disorder or disease, in particular caused or resulted in dysbiosis. Then, the invention also relates to a method for treating a disorder or a disease, for improving the general health of a subject and/or for modifying the composition of the microbiome, comprising administering a therapeutically effective amount of a composition of the invention to a subject in need thereof. It also relates to a composition of the invention for use for treating a disease or a disorder, for improving the general health of a subject and/or for modifying the composition of the microbiome. It also relates to a composition for use for the manufacture of a medicament, particularly for treating a disease or disorder, for improving the general health of a subject and/or for modifying the composition of the microbiome.

As used herein, the term “medicament” refers to any substance or composition with curative or preventive properties against a disorder or disease.

A “therapeutically effective amount” is an amount which, when administered to a subject, is sufficient to treat the targeted disease or disorder, or to produce the desired therapeutic effect. This amount may vary according to the disease and its severity, the physiological data and characteristics of the patient or subject to be treated (e.g. age, size, and weight), and the routes of administration.

Preferably, an “effective therapeutic amount” comprises 10³ to 10¹⁴ CFU (colony forming units), preferably 10⁶ to 10⁹ CFU of bacteria per ml or μg of the pharmaceutical composition.

In general, it is desirable to provide the subject with a dosage of the above bacteria or consortia in the range of from about 10³ to 10⁹ CFU/kg, preferably 10⁶ to 10⁹ CFU/kg (body weight of the subject), although a lower or higher dosage may be administered.

Alternatively, it can be desirable to provide the subject with a dosage of the composition of the invention in the range of from about 50 μg to 1 mg/kg, preferably 50 μg to 500 μg/kg, more preferably 50 μg to 250 μg/kg; even more preferably 50 μg to 100 μg/kg (body weight of the subject), although a lower or higher dosage may be administered.

Preferably, the composition is administered to the subject regularly, preferably between every day and every month, more preferably between every day and every two weeks, more preferably between every day and every week, even more preferably the composition is administered every day. In a particular embodiment, the composition is administered several times a day, preferably 2 or 3 times a day, even more preferably 3 times a day.

As used herein, the term “subject” or “patient” refers to an animal such as dogs, cats, horses, cows, pigs, sheep and non-human primates or non-mammals such as poultry, preferably a mammal, more preferably a human, including adult and child. In a particular embodiment, the composition of the invention comprises glycerol, so as to enhance butyrate production directly in the gut of a subject. Indeed, butyrate increase in the gut of the subject may result from i) the administration of the at least 10 mM butyrate comprised in the composition, but also ii) from the presence of glycerol that allows to increase butyrate production of butyrate producing bacteria comprised in the composition, once delivered in the gut. In this context, the composition can be particularly formulated so as the composition comprises a gastro-resistant form.

The pharmaceutical compositions may find use in a number of indications such as prophylaxis, treatment, prevention or delay of progression of a disease related to intestinal microbiome dysbalance or associated with microbiota dysbiosis. It is generally accepted that dysbiosis originates from an ecological dysbalance (e.g. based on trophism), characterized by disproportionate amounts or absence of bacteria strains in the microbiome of the patient which are essential for the establishment and/or maintenance of a healthy microbiome.

In particular, the composition of the invention can be used to treat pathologies involving bacteria of the human microbiome, preferably the intestinal microbiome, such as inflammatory or auto-immune diseases, cancers, infections or brain disorders. Indeed, some bacteria of the microbiome, without triggering any infection, can secrete molecules that will induce and/or enhance inflammatory or auto-immune diseases or cancer development.

In one embodiment, the disease or disorder to be treated by the composition according to the invention is selected from non-exhaustive group comprising cancer, intestinal infections, gastro-intestinal cancer, auto-immune diseases, infections such as caused by viruses or bacteria, ulcers, gastroenteritis, Guillain-Barre syndrome, graft versus host disease (GvHD), gingivitis and nosocomial infections.

More particularly, the disease or disorder to be treated by the composition according to the invention is selected from the group consisting of cancer, gastro-intestinal cancer, colorectal cancer (CRC), auto-immune disease, infection such as caused by viruses or bacteria, intestinal infection, ulcers, gastroenteritis, Guillain-Barre syndrome, graft versus host disease (GvHD), gingivitis, nosocomial infection, Clostridium difficile infection (CDI), infection by vancomycin resistant enterococci (VRE), post-infectious diarrhea, inflammatory bowel diseases (IBD), ulcerative colitis (UC) and Crohn's disease (CD).

In a particular embodiment, the disease or disorder to be treated is selected from Clostridium difficile infection (CDI), vancomycin resistant enterococci infection (VRE), post-infectious diarrhea, and inflammatory bowel diseases (IBD) including ulcerative colitis (UC) and Crohn's disease (CD), colorectal cancer (CRC), allo-HSCT associated diseases or Graft versus Host Disease (GvHD), preferably selected from Clostridium difficile infection (CDI), vancomycin resistant enterococci infections (VRE), post-infectious diarrhea, and inflammatory bowel diseases (IBD).

In a preferred embodiment, the disease or disorder to be treated is selected from inflammatory bowel diseases and Clostridium difficile infection.

In a particular embodiment, the subject has already received at least one line of treatment, preferably several lines of treatment, prior to the administration of the composition according to the invention.

The composition of the invention may be used as stand-alone-treatment (“mono therapy”) or treatment in combination with other pharmaceutics (“combination therapy”). The term “in combination” as used herein refers to the use of more than one therapy and does not restrict the order in which therapies are administered to a subject. The composition and the other treatments can be administered consecutively or simultaneously.

In particular, the composition of the invention can be used in combination with one or more cancer therapeutics, immunostimulatory agents, antibiotic agents, anti-inflammatory compounds, immunosuppressive compounds such as glucocorticoids, cytostatics or antibodies. Suitable cancer therapeutics are known per se and are preferably selected from the group of chemotherapy or radiotherapy agents, cancer immunotherapy agents (particularly checkpoint inhibitors, cancer vaccines, cytokines, cell therapy, CAR-T cells and dendritic cell therapy) and hormones angiogenesis inhibitors. Novel modalities applied in microbiome therapies such as therapies using phage, or phage like particles, DNA modifying, transferring or transcription silencing techniques and genetically modified bacteria can be used in combination with the composition of this invention.

### EXAMPLES

### Materials and Methods

### Bacterial Consortia

Bacterial strains were isolated from healthy donors using Hungate anaerobic culturing techniques (Bryant, 1972) and characterized for growth and metabolite production on M2GSC Medium (ATCC Medium 2857) and modifications thereof whereby the carbon sources glucose, cellobiose and starch were replaced by specific substrates including intermediate metabolites and fibers found in the human intestine.

The concentrations of the produced metabolites were quantified by refractive index detection HPLC (Thermo Scientific Accela™, ThermoFisher Scientific; HPLC-RI)). HPLC-RI analysis was performed using a SecurityGuard Cartridges Carbo-H (4×3.0 mm) (Phenomenex, Torrence, USA) as guard-column connected to a Rezex ROA-Organic Acid H+ column (300×7.8 mm) (Phenomenex). Bacteria cultures to be analyzed were centrifuged at 14.000-x g for 10 min at 4° C. Filter-sterilized (0.45 μL) supernatants were analyzed. Injection volume for each sample was 40 lpL. HPLC-RI was run at 40° C. with a flow rate of 0.4 mL/min and using H2SO4 (10 mM) as eluent. Peaks were analyzed using AgilentEzChrome Elite software (Version: 3.3.2 SP2, Agilent Technologies, Inc. Pleasanton, USA). Clusters were formed based on substrate usage and metabolite production.

Functional groups were defined as combinations of substrate-utilization and metabolite-production. Nine strains were selected within those clusters in order to assemble the core intestinal carbohydrate metabolism and result in an exclusive production of end metabolites (acetate, propionate and butyrate), without accumulation of intermediate metabolites (formate, succinate, lactate).

As outlined above, the combination of functional groups represented by one or more bacteria strains as disclosed herein is chosen to:
Degrade the main energy sources in the gut including fibers and intermediate metabolites (all groups, A1-A9).
Protect anaerobiosis by reduction of the eventual O2 through respiration (A3).
Produce the main end metabolites found in the intestine (A1, A2, A3, A4, A5, A9).
Prevent the enrichment of intermediate metabolites (A5, A6, A7, A8, A9) independent of the composition of the recipient's microbiome.

For group (A1), Ruminococcus bromii was cultivated in YCFA medium (Duncan, Hold, Harmsen, Stewart, & Flint, 2002) for 48 hours using the Hungate technique (Bryant, 1972) resulting in the production of formate (>15 mM) and acetate (>10 mM) as quantified by HPLC-RI.

For group (A2), Faecalibacterium prausnitzii was cultivated in M2GSC medium (ATCC Medium 2857) for 48 hours using the Hungate technique (Bryant, 1972) resulting in the consumption of acetate (>10 mM) and in the production of formate (>20 mM) and butyrate (>15 mM) as quantified by HPLC-RI.

For group (A3), Lactobacillus rhamnosus was cultivated in MRS Broth (Oxoid) for 48 hours using the Hungate technique (Bryant, 1972) resulting in the production of lactate (>50 mM) and formate (>10 mM) as quantified by HPLC-RI.

For group (A4), Bifidobacterium adolescentis was cultivated in YCFA medium (Duncan et al., 2002) for 48 hours using the Hungate technique (Bryant, 1972) resulting in the production of acetate (>50 mM), formate (>15 mM) and lactate (>5 mM) as quantified by HPLC-RI.

For group (A5), Anaerotignum (former Clostridium) lactatifermentans was cultivated in modified M2-based medium (ATCC Medium 2857) supplemented with DL-lactate [60 mM] instead of a carbohydrate source for 48 hours using the Hungate technique resulting in the consumption of lactate (at least 10 mM) and in the production of propionate (>30 mM), acetate (>10 mM) as detected by HPLC-RI.

For group (A6), Eubacterium limosum was cultivated in YCFA medium (Duncan et al., 2002) for 48 hours using the Hungate technique (Bryant, 1972) resulting in the production of acetate (>10 mM) and butyrate (>5 mM) as quantified by HPLC-RI.

For group (A7), Collinsella aerofaciens was cultivated in YCFA medium (Duncan et al., 2002) for 48 hours using the Hungate technique resulting in the production of formate (>20 mM), lactate (>15 mM) and acetate (>15 mM) as quantified by HPLC-RI.

For group (A8), Phascolarctobacterium faecium was cultivated in M2-based medium (ATCC Medium 2857) supplemented with succinate (60 mM) as sole carbohydrate source for 48 hours using the Hungate technique (Bryant, 1972) resulting in the full consumption of succinate (60 mM) and in the production of propionate (60 mM) as quantified by HPLC-RI.

For group (A9)—only part of PB002, Blautia hydrogenotrophica was cultivated in anaerobic AC21 medium (Leclerc, Bernalier, Donadille, & Lelait, 1997) for >75 hours using the Balch type tubes resulting in the production of acetate (>20 mM) as quantified by HPLC-RI, and consumption of hydrogen.

TABLE 1
Composition of the consortium PB002:

| Bacterial strain | Functional group |
|---|---|
| R. bromii | A1 |
| F. prausnitzii | A2 |
| Lb. rhamnosus | A3 |
| B. adolescentis | A4 |
| A. lactatifermentans | A5 |
| E. limosum | A6 |
| C. aerofaciens | A7 |
| P. faecium | A8 |
| B. hydrogenotrophica | A9 |

TABLE 2
Composition of the consortium PB010:

| Bacterial strain | Functional group |
|---|---|
| R. bromii | A1 |
| F. prausnitzii | A2 |
| Lb. rhamnosus | A3 |
| B. adolescentis | A4 |
| A. lactatifermentans | A5 |
| E. limosum | A6 + A9 |
| C. aerofaciens | A7 |
| P. faecium | A8 |

The combination of strains from the functional groups (A1)-(A9) encompass key functions of the microbiome and results, if cultured together, in a trophic chain analog to the healthy intestinal microbiome in its capacity to exclusively produce end metabolites from complex carbohydrates without accumulation of intermediate metabolites.

### Culture Conditions

A previously validated model for anaerobic intestinal fermentations (Zihler et al., 2013) was adapted using simplified medium based on YCFA (DSMZ Media No. 1611).

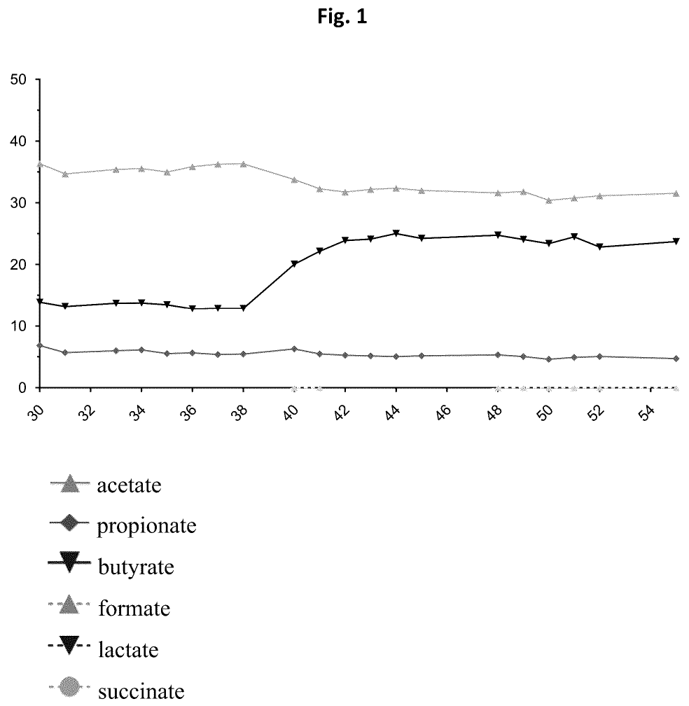

GMP009-Medium used for culturing PB002 (FIG. 1): Thereby, the 5 g/L of glucose that are the carbon source in YCFA were replaced by 3.25 g/L of sodium succinate (Sigma Aldrich), 2.25 g/L of maize starch (Roquette), and 2 g/L of D-lactose monohydrate (Sigma Aldrich). A 300 ml/500 ml bioreactor (Infors HT) was inoculated with a mix of overnight cultures of strains and inoculated anaerobically (0.7% v/v).

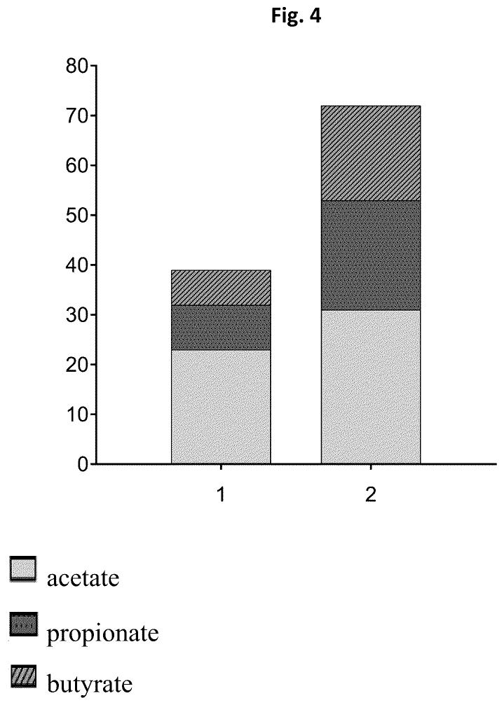

GMP007-Medium with glycerol for culturing PB010 (FIG. 4 (Bar Nr.(2)): The same carbon sources as specified above (GMP009-Medium) with the addition of 30 mM glycerol, which corresponds to 2.76 g/L medium. This medium was only fed throughout continuous operation of the bioreactor.

PBMF009-Medium used for PB010 (FIG. 4 (Bar Nr. (1)) Thereby, the 5 g/L of glucose that are the carbon source in YCFA were replaced by 3 g/L of cellobiose (Sigma Aldrich), 2 g/L of fructo-oligosaccharides (FB97, Cosucra), 3 g/L of soluble potato starch (Sigma Aldrich), and 4 g/L of pea starch (Roquette). A 300 ml/500 ml bioreactor (Infors HT) was inoculated with a mix of overnight cultures of strains and inoculated anaerobically (0.7% v/v).

The bioreactor was consecutively operated at pH 6.0 for 24 h in order to allow growth of primary degraders and subsequent consumption of the produced intermediate metabolites. Growth was monitored by base consumption and optical density. Metabolites were monitored using HPLC-RI as described above. After the first batch-fermentation, new medium was fed by removing half of total volume and refilling with medium to the original volume of 300 ml/500 ml in the bioreactor. After the second batch fermentation cycle the metabolic profile did not contain any intermediate metabolites and >30 mM acetate and >5 mM of propionate and butyrate each. From the end of the second batch fermentation on, the bioreactor was operated continuously at a volume of 300 ml/500 ml, a flow rate of 6 or 10 or 25 ml/h and a pH of 6.0 Subsequently, a stable metabolic profile established within 7 days after inoculation containing exclusively the desired end metabolites of acetate, propionate and butyrate without detection of intermediate metabolites showing constant production of all desired metabolites without washout of any functional group.

### Quantification of Bacterial Strains

To test maintenance of all bacterial strains of exemplary consortia PB002 and PB010 in the culture over time, qPCR quantification of the single strains of each consortium was performed using the primers listed in Table 3.

TABLE 3
List of primers used in qPCR

| Bacteria strains | Primer FW 5′-3′ | Primer RV 5′-3′ |
|---|---|---|
| R. bromii | CGCGT GAAGG ATGAA GGTTT TC | TCAGT TAAAG CCCAG CAGGC |
| | (SEQ ID NO: 1) | (SEQ ID NO: 2) |
| F. prausnitzii | CGCGG TAAAA CGTAG GTCAC A | CTGGG ACGTT GTTTC TGAGT TT |
| | (SEQ ID NO: 3) | (SEQ ID NO: 4) |
| L. rhamnosus | GGAAT CTTCC ACAAT GGACG CA | CATGG AGTTC CACTG TCCTC TT |
| | (SEQ ID NO: 5) | (SEQ ID NO: 6) |
| B. adolescentis | GTCCATCG CTTAACGG TGGATC | ACCAC CTGTG AACCC GC |
| | (SEQ ID NO: 7) | (SEQ ID NO: 8) |
| A. lactatifermentans | GCACT CCACC TGGGG AGT | CAACC TTCCT CCGGG TTATC CA |
| | (SEQ ID NO: 9) | (SEQ ID NO: 10) |
| E. limosum | GGCTT GCTGG ACAAA TACTG | CTAGG CTCGT CAGAA GGATG |
| | (SEQ ID NO: 11) | (SEQ ID NO: 12) |
| C. aerofaciens | GGTAG GGGAG GGTGG AAC | GCGGT CCCGC GTGGG TT |
| | (SEQ ID NO: 13) | (SEQ ID NO: 14) |
| P. faecium | GGAGT GCTAA TACCG GATGT GA | CCGTG GCTTC CTCGT TTACT |
| | (SEQ ID NO: 15) | (SEQ ID NO: 16) |
| B. hydrogenotrophica | CGTGA AGGAA GAAGT ATCTC GGTA | TCAGT TACCG TCCAG CAGGC C |
| | (SEQ ID NO: 17) | (SEQ ID NO: 18) |
| All bacteria | GTGST GCAYG GYTGT CGTCA | ACGTC RTCCC CRCCT TCCTC |
| | (SEQ ID NO: 19) | (SEQ ID NO: 20) |

*) sources:
(1) DECIPHER database;
(2) Wang et al. (1996),
(3) Maeda et al., (2003)

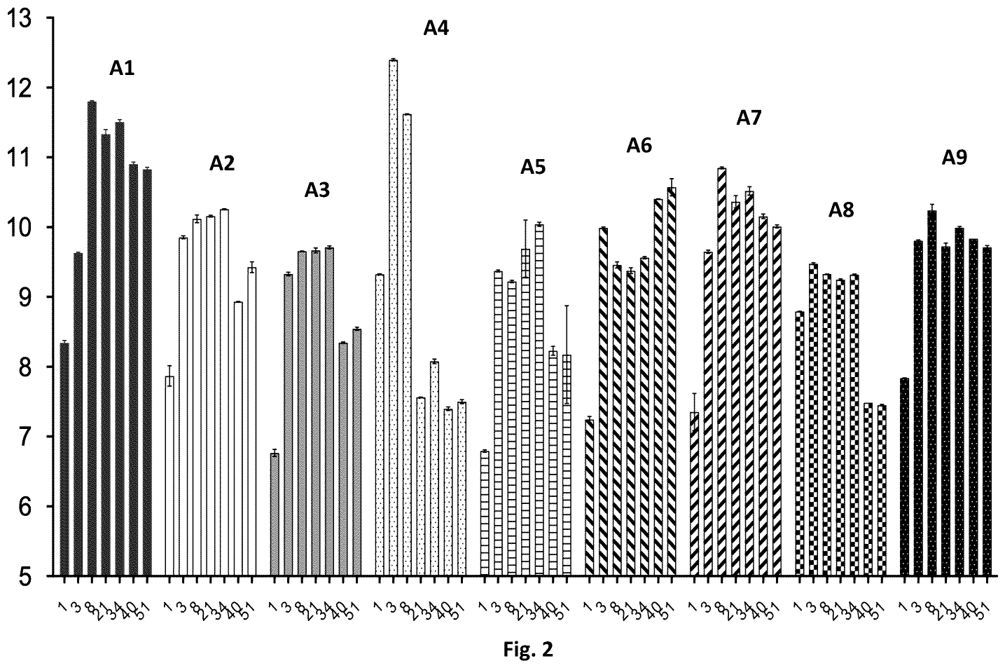

DNA from pellets of the fermentation effluent was extracted using the FastDNA™ SPIN Kit for Soil (MP Bio). Genomic DNA extracts were 10-fold diluted using DNA-free H2O. qPCRs were performed using Mastermix SYBR® green 2× and LowRox (Kapa Biosystems), primers (10 μM) and DNA-free H2O were used in a ABI 7500 FAST thermal cycler (Applied Biosystems) as recommended by the producer and quantified using standards of amplified whole 16S rRNA gene amplicon sequences of the strains cloned into the pGEMT easy vector (Promega, Madison WI, USA). Amplification of the whole 16S rRNA gene was performed with a combination of whole 16S rRNA gene amplification primers using one forward and one reverse primer from the primers listed in Table 4. qPCR quantification of the single strains is shown in copies of genomic 16S rRNA gene per ml of culture (FIG. 2).

TABLE 4

| | | Orientation of the |
|---|---|---|
| | | Primer on 16S rRNA |
| Name *) | Sequence 5′-3′ **) | Gene Sequence 5′-3′ |
| 518R 5) | ATTAC CGCGG CTGCT GG (SEQ ID NO: 21) | Reverse |
| 1392R 1) | ACGGG CGGTG TGTRC (SEQ ID NO: 22) | Reverse |
| 1412R 2) | CGGGT GCTNC CCACT TTCAT G (SEQ ID NO: 23) | Reverse |
| 1492R 4) | GNTAC CTTGT TACGA CTT (SEQ ID NO: 24) | Reverse |
| 1492R.E 1) | TACGG YTACC TTGTT ACGAC TT (SEQ ID NO: 25) | Reverse |
| 1525R 1) | AAGGA GGTGW TCCAR CC (SEQ ID NO: 26) | Reverse |
| F8 4) | AGAGT TTGAT CMTGG CTC (SEQ ID NO: 27) | Forward |
| F15 2) | GATTC TGGCT CAGGA TGAAC G (SEQ ID NO: 28) | Forward |
| F27 1) | AGAGT TTGAT CMTGG CTCAG (SEQ ID NO: 29) | Forward |
| F518 5) | CCAGC AGCCG CGGTA ATACG (SEQ ID NO: 30) | Forward |

*) sources:
1) Lane, 1991,
2) Kaufmann et al., 1997,
4) Mosoni et al., 2007),
5) Muyzer et al., 1993
**) nucleic codes as defined in IUPAC nucleotide code, particularly: N = any base, R = A or G.

### Preservation of Bacterial Consortia

The effluent of the consortia continuously fermented for at least 7 days was anaerobically mixed 1:1 with an anaerobic cryoprotective medium containing 60% glycerol and 40% of the dispersing medium previously described above (see “Culture conditions”). The cryoprotected formulation was snap cryopreserved using liquid nitrogen and stored at −20° C./−80° C. for at least 1 month. The stored effluents were used for efficacy tests in a mouse model as described below.

### Results

### Example 1

The consortium PB002 was continuously cultured in a bioreactor during 55 days. Metabolite concentrations were measured daily before and after glycerol supplementation (addition of 30 mM glycerol) at day 38. Results are presented in FIG. 1 and show that supplementation of glycerol at day 38 enhanced butyrate production immediately with no accumulation of the intermediate metabolites formate, lactate, or succinate.

During this culture, absolute abundances of all bacterial strains of the consortium were also quantified using qPCR. Results are shown in FIG. 2 and are indicated in log 10 copies of the 16S rRNA gene/ml of culture for each strain. It was thus demonstrated that glycerol supplementation positively affects abundance of butyrate producing strains of group A6.

### Example 2

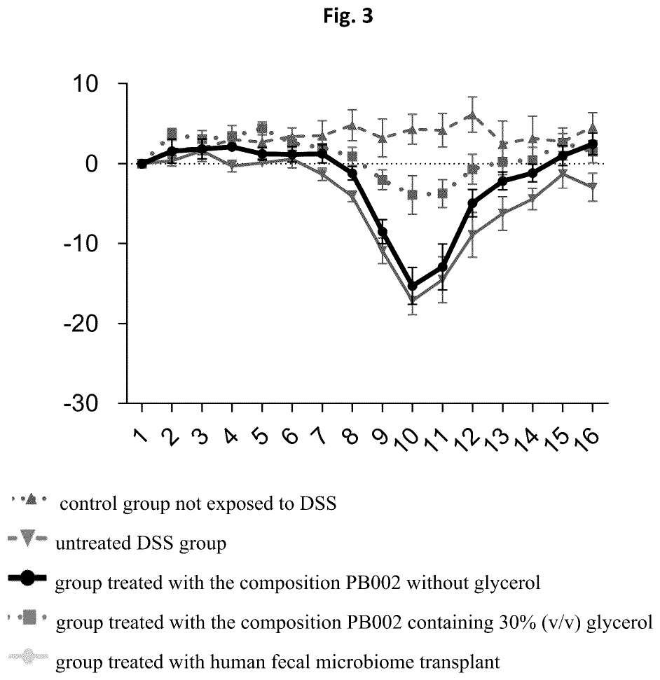

To compare the in vivo efficacy of fresh reactor effluent operated with PB002 and cryopreserved PB002 an acute DSS (dextran sodium sulfate) mouse model was performed. This is a well-accepted model for dysbiosis, causing colitis. To induce acute colitis, 12-15 weeks old C57/B6 mice were treated with 3% DSS in drinking water for 7 days. The DSS induced intestinal barrier rupture leads to increased severe diarrhea, intestinal inflammation and consecutive weight loss (expressed in % of initial weight at day 1 of every group in FIG. 3). After 5-7 days of having access to normal drinking water, mice recover spontaneously and return from their dysbiotic, inflammatory state to normal weight.

All treatment groups were gavaged on 3 consecutive days (day 8, 9 and 10) with 200 μl of the respective suspensions. Groups of 4-5 mice were made separated into
control group that was not exposed to DSS,
the untreated DSS group,
the group treated with freshly produced PB002 reactor effluent (no glycerol)—“PB002 fresh”,
the group treated with cryopreserved PB002 (containing 30% v/v glycerol—“PB002 frozen”.

Weight was measured daily until sacrifice on day 16. Mice treated with frozen PB002, containing 30% v/v of glycerol showed faster weight recovery starting at the third day of treatment compared to fresh PB002 effluent. The data presented in FIG. 3 therefore show that the consortium supplemented with glycerol can restore a dysbiotic microbiota more efficient than the same consortium without glycerol.

### Example 3

The consortium PB010 was continuously cultured (i) in a bioreactor comprising a culture medium without glycerol and (ii) in a bioreactor comprising a culture medium supplemented with glycerol. Absolute metabolite concentrations were measured at day 8 after inoculation. Results showed that there is no accumulation of succinate, lactate or formate and that the supplementation of glycerol stimulates butyrate production of the consortium (FIG. 4).

### Example 4

The bacterial consortium PB002 and the bacterial consortium PB010 were continuously cultured in bioreactors. Relative end metabolite concentrations were determined on day 2 and 8 of co-cultivation.

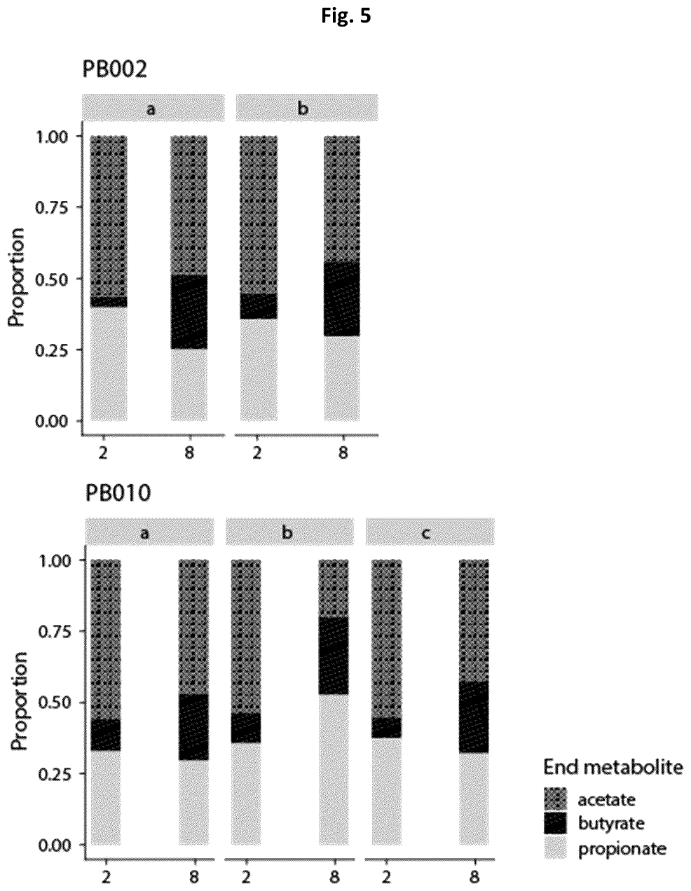

All bioreactors were cultivated for 48 hours in a medium without glycerol. After day 2, glycerol was supplemented, followed by an increase in butyrate production as shown on day 8. The results show that the addition of glycerol to the growth medium leads to a reproducible and repeatable increase in butyrate production for two different consortia (FIG. 5).

### REFERENCES

Bryant, M. P. (1972). Commentary on the Hungate technique for culture of anaerobic bacteria. The American Journal of Clinical Nutrition, 25(12), 1324-1328.
Duncan, S. H., Hold, G. L., Harmsen, H. J. M., Stewart, C. S., & Flint, H. J. (2002). Growth requirements and fermentation products of Fusobacterium prausnitzii, and a proposal to reclassify it as Faecalibacterium prausnitzii gen. nov., comb. nov. International Journal of Systematic and Evolutionary Microbiology, 52(6), 2141-2146.
Kaufmann, P., Pfefferkorn, A., Teuber, M., & Meile, L. (1997). Identification and quantification of Bifidobacterium species isolated from food with genus-specific 16S rRNA gene-targeted probes by colony hybridization and PCR. Applied and Environmental Microbiology, 63(4), 1268-1273.
Lane, D. J. (1991). 16S/23S rRNA Sequencing. In E. Stackebrandt & M. Goodfellow (Eds.), Nucleic Acid Techniques in Bacterial Systematics (Chapter 6 pp. 115-148). Sussex: John Wiley and Sons.
Leclerc, M., Bernalier, A., Donadille, G., & Lelait, M. (1997). H2/CO2 metabolism in acetogenic bacteria isolated from the human colon. Anaerobe, 3(5), 307-315.
Maeda, H., Fujimoto, C., Haruki, Y., Maeda, T., Kokeguchi, S., Petelin, M., Takashiba, S. (2003). Quantitative real-time PCR using TaqMan and SYBR Green for Actinobacillus actinomycetemcomitans, Porphyromonas gingivalis, Prevotella intermedia, tetQ gene and total bacteria. FEMS Immunology and Medical Microbiology, 39(1), 81-86.
Mosoni, P., Chaucheyras-Durand, F., Bera-Maillet, C., & Forano, E. (2007). Quantification by real-time PCR of cellulolytic bacteria in the rumen of sheep after supplementation of a forage diet with readily fermentable carbohydrates: effect of a yeast additive. Journal of Applied Microbiology, 103(6), 2676-85.
Muyzer, G., de Waal, E. C., & Uitterlinden, A. G. (1993). Profiling of complex microbial populations by denaturing gradient gel electrophoresis analysis of polymerase chain reaction-amplified genes coding for 16S rRNA. Applied and Environmental Microbiology, 59(3), 695-700.
Wang, R. F., Cao, W. W., & Cerniglia, C. E. (1996). PCR detection and quantitation of predominant anaerobic bacteria in human and animal fecal samples. Applied and Environmental Microbiology, 62(4), 1242-1247.
Zihler, A., Fuentes, S., Dostal, A., Payne, A. N., Vazquez Gutierrez, P., Chassard, C., & Lacroix, C. (2013). Novel Polyfermentor Intestinal Model (PolyFermS) for Controlled Ecological Studies: Validation and Effect of pH. PloS One, 8(10).

1-26. (canceled)

27. A pharmaceutical or nutraceutical composition comprising a consortium of bacteria comprising:
a) at least one bacterial strain producing lactate;
b) at least one bacterial strain consuming lactate;
c) at least one bacterial strain producing butyrate, the production of butyrate of said bacterium being increased by at least 10% in the presence of glycerol; and
d) at least 10 mM butyrate.

28. The pharmaceutical or nutraceutical composition according to claim 27, which further comprises at least 5% glycerol.

29. The pharmaceutical or nutraceutical composition according to claim 27, wherein the butyrate producing bacterium is also a lactate consuming bacterium.

30. The pharmaceutical or nutraceutical composition according to claim 27, wherein the bacterial strain producing butyrate is selected from the genera Eubacterium, Roseburia, Coprococcus, Faecalibacterium, Anaerostipes and Clostridium.

31. The pharmaceutical or nutraceutical composition according to claim 30, wherein the bacterial strain producing butyrate is selected from:
the genus Eubacterium and is Eubacterium limosum, Eubacterium rectale, Eubacterium hallii or Eubacterium ramulus;
the genus Roseburia and is Roseburia spp., Roseburia intestinalis, Roseburia hominis, Roseburia inulinivorans, or Roseburia faecis;
the genus Coprococcus and is Coprococcus catus Coprococcus eutactus, or Coprococcus comes;
the genus Faecalibacterium and is Faecalibacterium prausnitzii;
the genus Anaerostipes and is Anaerostipes caccae or Anaerostipes hadrus; or
the genus Clostridium and is Clostridium indolis.

32. The pharmaceutical or nutraceutical composition according to claim 27, wherein the composition further comprises:
(i) at least one lactate producing bacterium selected from the genera Lactobacillus, Streptococcus, Escherichia, Lactococcus, Enterococcus, Bifidobacterium, Collinsella, and Roseburia; and
at least one lactate consuming bacterium selected from the genera Anaerostipes, Eubacterium, Clostridium, Propionibacterium, Veillonella, Coprococcus and Megasphaera; and
(ii) optionally:
at least one bacterial strain selected from the genera Ruminococcus, Dorea, Clostridium and Eubacterium (A1);
at least one bacterial strain selected from the genera Phascolarctobacterium, Flavonifractor and Dialister (A8);
at least one bacterial strain selected from the genera Acetobacterium, Clostridium, Eubacterium, Moorella, Methanobrevibacter, Methanomassiliicoccus and Sporomusa (A9);
at least one bacterial strain selected from the genera Alistipes, Bacteroides, Barnesiella, Clostridium, Ruminococcus and Prevotella (A10);
at least one bacterial strain selected from the genera Clostridium, Coprococcus, Eubacterium, Flavonifractor and Flintibacter (A11);
at least one bacterial strain selected from the genera Bacteroides, Barnesiella, Bifidobacterium, Clostridium, Enterococcus, Faecalibacterium, Lactobacillus and Ruminococcus (A12);
at least one bacterial strain selected from the genera Anaerostipes, Blautia, Clostridium and Faecalibacterium (A13);
at least one bacterial strain selected from the genera Bacteroides, Bifidobacterium, Blautia, Clostridium, Faecalibacterium, Lactobacillus, Prevotella and Ruminococcus (A14); and
at least one bacterial strain selected from the genera Akkermansia, Bacteroides, Bifidobacterium and Ruminococcus (A15).

33. The pharmaceutical or nutraceutical composition according to claim 27, wherein the composition does not comprise Blautia hydrogenotrophica.

34. The pharmaceutical or nutraceutical composition according to claim 27, wherein the composition comprises:
(i) Eubacterium limosum and/or Faecalibacterium prausnitzii as butyrate producers,
Lactobacillus rhamnosus, Collinsella aerofaciens and/or Bifidobacterium adolescentis as lactate producers, and
Anaerotignum (former Clostridium) lactatifermentans and/or Eubacterium limosum as lactate consumers; and
(ii) at least one bacterium selected from the group consisting of Ruminococcus bromii, Phascolarctobacterium faecium;
and
(iii) optionally Bacteroides xylanisolvens.

35. The pharmaceutical or nutraceutical composition according to claim 27, wherein the composition comprises Ruminococcus bromii (A1), Faecalibacterium prausnitzii (A2), Lactobacillus rhamnosus (A3), Bifidobacterium adolescentis (A4), Anaerotignum (former Clostridium) lactatifermentans (A5), Eubacterium limosum (A6 and A9), Collinsella aerofaciens (A7) and Phascolarctobacterium faecium (A8) and optionally Bacteroides xylanisolvens (A10).

36. The pharmaceutical or nutraceutical composition according to claim 27, which is free of, or essentially free of one or more of succinate, formate and lactate; and/or which further comprises propionate and/or acetate.

37. A method of treating a disease comprising the administration of a pharmaceutical or nutraceutical composition according to claim 27 to a subject in need of treatment, wherein the disease is selected from the group consisting of cancer, gastro-intestinal cancer, colorectal cancer (CRC), intestinal infections, auto-immune disease, viral infections, bacterial infections, ulcers, gastroenteritis, Guillain-Barre syndrome, graft versus host disease (GvHD), gingivitis, nosocomial infection, Clostridium difficile infection (CDI), infection by vancomycin resistant enterococci (VRE), post-infectious diarrhea, inflammatory bowel diseases (IBD), ulcerative colitis (UC) and Crohn's disease (CD).

38. A method for producing butyrate, said method comprising culturing a butyrate producing bacterium in a culture medium comprising glycerol, wherein the butyrate producing bacterium is comprised in a consortium of bacteria according to claim 27, and optionally recovering butyrate, wherein the production of butyrate of said bacterium is increased by at least 10% in the presence of glycerol.

39. The method according to claim 38, wherein glycerol is added to the culture medium at a concentration of more than 5% (v/v) prior or during cultivation.

40. The method according to claim 38, wherein butyrate, and optionally bacterial cells, are recovered when the concentration of butyrate in the culture medium is above 10 mM.

41. A pharmaceutical or nutraceutical composition comprising a viable butyrate producing bacterium in a consortium of bacteria, at least 10 mM butyrate, and optionally glycerol, wherein butyrate and the butyrate producing bacterium are obtained by a method according to claim 38, said consortium of bacteria comprising:
a) at least one bacterial strain producing lactate;
b) at least one bacterial strain consuming lactate; and
c) at least one bacterial strain producing butyrate, the production of butyrate of said bacterium being increased by at least 10% in the presence of glycerol.

42. The pharmaceutical or nutraceutical composition according to claim 41, which is free of, or essentially free of one or more of succinate, formate and lactate; and/or which further comprises propionate and/or acetate.

43. The pharmaceutical or nutraceutical composition according to claim 41, which comprises at least 5% of glycerol.

44. A method of treating a disease comprising the administration of a composition according to claim 41 to a subject in need of treatment, wherein the disease is selected from the group consisting of cancer, gastro-intestinal cancer, colorectal cancer (CRC), intestinal infections, auto-immune disease, viral infections, bacterial infections, ulcers, gastroenteritis, Guillain-Barre syndrome, graft versus host disease (GvHD), gingivitis, nosocomial infection, Clostridium difficile infection (CDI), infection by vancomycin resistant enterococci (VRE), post-infectious diarrhea, inflammatory bowel diseases (IBD), ulcerative colitis (UC), and Crohn's disease (CD).
